SEQUENCE LISTING (7 sequences)

SEQ ID NO: 1 - DNA, 441 bp, Saccharomyces cerevisiae
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 2 - DNA, 960 bp, Homo sapiens
aagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaagccggacacagcctggagctgattgagcccctcatcaagttccaggtgggactgaagaagctgaacttgcatgaggaggagcatgtcctgctcatggccatctgcatcgtctccccagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctga

SEQ ID NO: 3 - DNA, 100 bp, Saccharomyces cerevisiae
caaggcggagtactgtcctccgggctggcggagtactgtcctccggcaaggtcggagtactgtcctccgacactagaggtcggagtactgtcctccgacg

SEQ ID NO: 4 - DNA, 152 bp, Artificial Sequence
  Minimal Thymidin kinase promoter
gtggccgccccgactgcatctgcgtgttcaaattcgccaatgacaagacgctgggcggggtttgtgtcatcatagaactaaagacatgcaaatatatttcttccggggacaccgccagcaaacgcgagcaacgggccacggggatgaagcag

SEQ ID NO: 5 - DNA, 720 bp, Artificial Sequence
  d2eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaag

SEQ ID NO: 6 - DNA, 225 bp, Artificial Sequence
  Right Inverted Repeats (T2)
agtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 7 - DNA, 225 bp, Artificial Sequence
  Laft Inverted Repeats (T2)
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacact

(19) United States
(12) Patent Application Publication
(10) Pub. No.: US 2011/0265192 A1
(43) Pub. Date: Oct. 27, 2011

(54) NUCLEAR RECEPTOR SENSOR SYSTEM IN TRANSGENIC ANIMAL

(75) Inventors: Lars Axel Bolund, Skodstrup (DK); Karsten Kristiansen, Broby (DK); Jacob Giehm Mikkelsen, Silkeborg (DK); Nicklas Heine Staunstrup, Aarhus N (DK)

(73) Assignees: SYDDANSK UNIVERSITET, Odense M (DK); AARHUS UNIVERSITET, Arhus C (DK)

(21) Appl. No.: 13/062,028

(22) PCT Filed: Sep. 3, 2009
(86) PCT No.: PCT/DK2009/050225

§ 371 Date: Jun. 22, 2011

(30) Foreign Application Priority Data
Sep. 5, 2008 (DK) PA 2008 01241

(51) Int. Cl. G01N 33/00; C12N 15/873; A01K 67/00; A01K 67/027; C12N 5/10; C12Q 1/68

(52) U.S. Cl. 800/3; 435/325; 435/6.13; 800/13; 800/17; 800/18; 800/14; 800/21; 435/6.1

(57) ABSTRACT

A sensor system for detecting the activation of specific nuclear receptors in a tissue of an animal is provided. The nuclear receptor sensor system comprises a sensor component comprising a nuclear receptor or part thereof coupled to a DNA-binding domain, and a reporter component comprising a reporter gene. Transgenic animals, such as a transgenic pig is provided, which comprises the components of the nuclear receptor sensor system in its genome. Also methods of producing the transgenic animal is provided as well as use of the transgenic animal for evaluating the activity of a nuclear receptor in vivo.

### FIELD OF INVENTION

The present invention relates to method for evaluating a physical or chemical agent for its effect on a tissue in a transgenic animal, such as a transgenic pig.

### BACKGROUND OF INVENTION

Transcription factors belonging to the superfamily of nuclear receptors play pivotal roles in cellular growth, differentiation and apoptosis. Nuclear receptors are involved in the regulation of gene expression by activating the expression of specific target genes. Thus, the activation of nuclear receptors leads to changes in gene expression, and are suspected be involved in the route towards developing a range of disorders. Nuclear receptors are a primary target for a range of drugs. It is well established that classical nuclear hormone receptors such as the retinoic acid and vitamin D receptors regulate cellular fate, and in keratinocytes, retinoids and vitamin D analogs have gained widespread use in the treatment of epidermal disorders. Recently members of the so-called peroxisome proliferator-activated receptor (PPAR) family have been implicated in the control of keratinocyte growth and differentiation, and it has been suggested that drugs targeting PPARs should be considered as potential skin therapeutic agents

Given the impact of nuclear receptors for cellular growth, differentiation and apoptosis, a method for detecting the activation of specific nuclear receptors is extremely valuable. Such a method may be used to evaluate the effect of exposing a specific tissue to an agent that potentially induces nuclear receptor mediated gene activation. This would be an invaluable tool in dissecting the mechanisms behind a large range of disorders that are associated with activation of nuclear receptors.

A method for detection of the activation of a nuclear receptor has been employed in molecular biological studies. This method makes use of two nucleic acid cassettes: one cassette comprising the sensor component and one cassette comprising the reporter component. Previously, this nuclear receptor sensor system, however, has only been employed in single cell cultures. In the present invention, the nuclear receptor sensor system is functionally incorporated into the genome of a transgenic animal, such as a transgenic mouse or pig.

Integration of the nuclear receptor sensor system into the genome of a transgenic animal allows for in vivo temporal-spatial analysis of agents, which potentially affect nuclear receptors. For example integration of the nuclear receptor sensor system in the skin of a transgenic animal allows for studies of the penetration of applied pharmaceuticals or xenobiotics through the layers of the skin.

Both animals and humans are exposed to an immense amount of different physical and chemical agents, such as UV radiation, xenobiotics and food additives every day, many of these through contact with skin or the digestive tract. However, the potential health implications of this exposure have not been characterized for the vast majority of those agents, and hence, there is a need to assess to what extent they penetrate the skin, intestinal wall or other relevant tissue. It is known that a considerable number of agents are able to influence the activity of members of the nuclear receptor family, and hence there is a need to develop experimental systems that allows efficient and cost effective methods for determination of the ability of a given physical or chemical agent to penetrate a specific tissue and activate nuclear receptors.

Because of their complex structure, human skin models are difficult to obtain. Epidermal penetration has been widely assessed using human skin explants. However, it is difficult to obtain sufficient human skin for explant studies, and furthermore, viable human skin explants suffer from a number of drawbacks such as considerable variability in sample size, shape and quality. Alternatives to human skin explants are reconstituted human skin generated from isolated human keratinocytes. Reconstituted epidermal models are elegant, useful and practical tool, and have been demonstrated to mimic many of the molecular and biophysical properties of human epidermis. However, the barrier function of reconstituted epidermis is reduced compared to normal epidermis and human skin explants. A useful alternative is pig skin, which has proven to be a good model for human skin and has been recommended for dermal absorption studies (OCDE Guidance document for the conduct of skin absorption studies; Series on testing and assessment, No 28.). The organization of pig skin resembles human skin, and it is predicted that the generation of skin sensor pig strains in combination with the advanced fluorescence-based protocols will prove of importance for basic as well as more applied in situ purposes (penetration of test substances and xenobiotics). By use of the recent progress in pig cloning and gene transfer technology it is possible to integrate the nuclear receptor sensor system into a pig to produce cloned transgenic pigs in which the skin harbors the sensor systems, and hence, can by use for in vivo penetration studies. Such a transgenic animal will provide an important technology to study skin penetration of pharmaceuticals and xenobiotics. Such knowledge will be of importance in relation to drug delivery to the skin, and for risk evaluation of xenobiotics.

### SUMMARY OF INVENTION

The present invention relates to a method that allows for the evaluation of the effect of an agent on a tissue. The invention also applies as a method for evaluation the efficiency of an additional compound, which is applied to the tissue prior to that agent, to minimize or maximize the effect of said physical or chemical agent on said tissue.

In one aspect, the present invention relates to a method for evaluating the effect of an agent in a tissue of an animal comprising a) providing a transgenic animal, comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence, b) administering said agent to said animal, and c) evaluating the transcriptional and/or translational expression product of the nucleic acid sequence encoding the reporter polypeptide, wherein an alteration of said expression product prior to and after step (b) is indicative of an effect on said tissue.

In another aspect, the invention relates to a method for testing a compound for the ability to alter the effects of an agent in a tissue of an animal comprising a) administering said compound to a transgenic animal comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence, b) administering said agent to said transgenic animal, and c) evaluating the transcriptional and/or translational expression product of the nucleic acid sequence encoding the reporter polypeptide, wherein a difference in the amount of said expression product in the presence and absence of said compound is indicative of said compound being able to alter the effect of said agent in said tissue.

In a third aspect, the invention relates to a transgenic animal comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence.

In a fourth aspect, the present invention pertains to a cell line derived from the transgenic animal according to the present invention.

The present invention also comprises a non-human transgenic animal, which comprises the sensor and/or reporter cassette of the present invention. Thus, in one aspect, the present invention relates to a transgenic animal comprising

i. at least one nucleic acid sequence encoding a nuclear receptor or part thereof and a DNA binding domain or part thereof, and

ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or

iii. the transcriptional or translational products of any of said nucleic acid sequences,

The transgenic animal is in one embodiment selected from the group consisting of pig, minipig, micropig, mouse, rat, non-human primate and rodent, and is preferably a pig. The nuclear receptor is preferably Thyroid hormone receptor-α (TRα; NR1A1, THRA), Thyroid hormone receptor-β (TRβ; NR1A2, THRB), Retinoic acid receptor-α (RARα; NR1B1, RARA), Retinoic acid receptor-β (RARβ; NR1B2, RARB), Retinoic acid receptor-γ (RARγ; NR1B3, RARG), Peroxisome proliferator-activated receptor-α (PPARα; NR1C1, PPARA), Peroxisome proliferator-activated receptor-β/δ (PPARβ/δ NR1C2, PPARD), Peroxisome proliferator-activated receptor-γ (PPARγ; NR1C3, PPARG), Rev-ErbAα (Rev-ErbAα; NR1D1), Rev-ErbAp (Rev-ErbA(3; NR1D2), RAR-related orphan receptor-α (RORα; NR1F1, RORA), RAR-related orphan receptor-β (RORβ; NR1F2, RORB), Liver X receptor-α (LXRα; NR1H3), Liver X receptor-β (LXRβ; NR1H2), Farnesoid X receptor (FXR; NR1H4), Vitamin D receptor (VDR; NR1I1, VDR) (vitamin D), Pregnane X receptor (PXR; NR1I2), Constitutive androstane receptor (CAR; NR1I3), Hepatocyte nuclear factor-4-α (HNF4α; NR2A1, HNF4A), Hepatocyte nuclear factor-4-γ (HNF4γ; NR2A2, HNF4G), Retinoid X receptor-α (RXRα; NR2B1, RXRA), Retinoid X receptor-β (RXRβ; NR2B2, RXRB), Retinoid X receptor-γ (RXRγ; NR2B3, RXRG), Testicular receptor 2 (TR2; NR2C1), Testicular receptor 4 (TR4; NR2C2), Human homologue of the Drosophila tailless gene (TLX; NR2E1), Photoreceptor cell-specific nuclear receptor (PNR; NR2E3), Chicken ovalbumin upstream promoter-transcription factor I (COUP-TFI; NR2F1), Chicken ovalbumin upstream promoter-transcription factor II (COUP-TFII; NR2F2), 6: V-erbA-related (EAR-2; NR2F6), Estrogen receptor-α (ERα; NR3A1, ESR1), Estrogen receptor-β (ERβ; NR3A2, ESR2), Estrogen related receptor-α (ERRα; NR3B1, ESRRA), Estrogen related receptor-β (ERRβ; NR3B2, ESRRB), Estrogen related receptor-γ (ERRγ; NR3B3, ESRRG), Glucocorticoid receptor (GR; NR3C1) (Cortisol), Mineralocorticoid receptor (MR; NR3C2) (Aldosterone), Progesterone receptor (PR; NR3C3, PGR) (Sex hormones: Progesterone), Androgen receptor (AR; NR3C4, AR) (Sex hormones: Testosterone), Nerve Growth factor IB (NGFIB; NR4A1), Nuclear receptor related 1 (NURR1; NR4A2), Neuron-derived orphan receptor 1 (NOR1; NR4A3), Steroidogenic factor 1 (SF1; NR5A1), Liver receptor homolog-1 (LRH-1; NR5A2), Germ cell nuclear factor (GCNF; NR6A1), DAX1 (Dosage-sensitive sex reversal, adrenal hypoplasia critical region, on chromosome X, gene 1 (NR0B1)), Small heterodimer partner (SHP; NR0B2) or Nuclear receptors with two DNA binding domains (2DBD-NR), and more preferably the nuclear receptor is selected from the group consisting of vitamin D receptor, Liver X receptors, Retinoic Acid receptor, Retinoid X receptor, promiscuous pregnane X receptor and peroxisome proliferation activation receptors (PPARs), including PPARα, PPARβ/δ, PPARγ. In a preferred embodiment, the nuclear receptor or part thereof comprise a ligand binding domain of a nuclear receptor or a fragment thereof.

A preferred transgenic animal of the present invention comprises

a. at least one nucleic acid sequence encoding a fusion polypeptide, comprising PPARδ or part thereof coupled to yeast GAL4 DNA binding domain and/or

b. at least one nucleic acid sequence encoding β-galactosidase or part thereof.

The DNA binding domain of the transgenic animal of the present invention is preferably GAL4 DNA-binding domain, LexA DNA-binding domain, and/or any part thereof.

Moreover, the nuclear receptor or part thereof and DNA binding domain or part thereof is preferably expressed from an inducible and/or a tissue-specific promoter, such as a promoter, which is specific for a tissue selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and/or tumours. In a preferred embodiment, the tissue-specific promoter is keratin 14 enhancer/promoter.

The nucleic acid sequence encoding a detectable reporter nucleic acid transcript and/or reporter polypeptide or part thereof in one embodiment further comprises at least one yeast Gal4 upstream activation sequence (UASgal), bacterial LexA binding site and/or a part thereof. Moreover, the nucleic acid sequence encoding a detectable reporter transcript or polypeptide is preferably expressed from a heterologous and/or inducible promoter.

In a preferred embodiment, the nuclear receptor or part thereof and a DNA binding domain or part thereof are physically or chemically coupled, for example, the polypeptides are expressed preferably as a fusion peptide.

The expression of said nuclear receptor or part thereof and a DNA binding domain or part thereof preferably promotes expression of the reporter polypeptide.

The reporter transcript, polypeptide or fragment thereof preferably comprises a visually, optically or autoradiographically detectable product, and thus, the reporter polypeptide is in one embodiment selected from the group consisting of β-galactosidase, HcRed, DsRed, DsRed monomer, ZsGreen, AmCyan, ZsYellow, fire fly luciferase, lac Z, renilla luciferase, SEAP, enhanced green fluorescent protein (eGFP), d2EGFP, enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, human alpha-1-antitrypsin (hAAT) and/or fragments, modifications or functional variants thereof; in a preferred embodiment, the reporter polypeptide is β-galactosidase. Expression of reporter transcript or polypeptide is detectable by any suitable detection technique available to those of skill in the art, such as a technique selected from enzymatic or spectroscopic assays, confocal or multiphoton fluorescent microscopy, western blotting, imunostaining, Enzyme-linked immunosorbent assay (ELISA) as well as nucleic acid detection techniques such as northern blotting, southern blotting, polymerase chain reaction, primer extension and DNA array technologies.

In another aspect, the present invention relates to a method for evaluating the effect of an agent on the activity of a nuclear receptor in a tissue of a non-human animal, said method comprising

a. providing a non-human transgenic animal of the present invention as defined above,

b. administering an agent to said transgenic animal, and

c. detecting the expression of said nucleic acid sequence encoding a reporter nucleic acid transcript and/or reporter polypeptide or part thereof in said animal,

wherein the expression upon administration of said agent is indicative of the effect of said agent on the activity of a nuclear receptor in said tissue.

In yet another aspect the present invention relates to a method for testing a compound for the ability to alter an effect of an agent on the activity of a nuclear receptor in a tissue of a non-human animal comprising

a. providing a non-human transgenic animal of the present invention as defined above,

b. administering said compound to said transgenic animal,

c. administering said agent to said transgenic animal, and

d. detecting the expression of said nucleic acid sequence encoding a reporter nucleic acid transcript and/or reporter polypeptide or part thereof in said animal,

wherein the expression upon administration of said agent is indicative of the effect of said agent on the activity of a nuclear receptor in said tissue.

In the methods of the present invention, the expression of the nucleic acid sequence encoding a reporter nucleic acid transcript and/or reporter polypeptide or part thereof is detected in the presence and absence of said agent and/or compound. In preferred embodiments of the methods of the present invention, in the presence of said agent compared with the absence of said agent

a. an increase in the expression of said reporter transcript or polypeptide is indicative of a stimulatory effect of said agent on the activity of said nuclear receptor,

b. a decrease in the expression of said reporter transcript or polypeptide is indicative of an inhibitory effect of said agent on the activity of said nuclear receptor, and

c. an unchanged expression of said reporter transcript or polypeptide is indicative of said agent having no or little effect on the activity of said nuclear receptor.

Similarly, in other preferred embodiments of the methods of the present invention, in the presence of said compound compared with the absence of said compound

a. an increase in the effect of said agent on the expression of said reporter transcript or polypeptide is indicative of a stimulatory effect of said compound on the effect of said agent on the activity of said nuclear receptor,

b. a decrease in the effect of said agent on the expression of said reporter transcript or polypeptide is indicative of an inhibitory effect of said compound on the effect of said agent on the activity of said nuclear receptor, and

c. a little or unchanged effect of said agent on the expression of said reporter transcript or polypeptide is indicative of said compound having no or little effect on the effect of said agent on the activity of said nuclear receptor.

According to the methods of the present invention, expression of said nuclear receptor or part thereof and a DNA binding domain or part thereof promotes expression of said reporter polypeptide. Expression of the nucleic acid sequence encoding the reporter polypeptide is preferably detected by detecting the transcriptional and/or translational expression product of the nucleic acid sequence encoding the reporter polypeptide, for example, detection of the expression of said reporter transcript or polypeptide comprises detection by any technique selected from enzymatic or spectroscopic assays, confocal or multiphoton fluorescent microscopy, western blotting, imunostaining, Enzyme-linked immunosorbent assay (ELISA) as well as nucleic acid detection techniques such as northern blotting, southern blotting, polymerase chain reaction, primer extension and DNA array technologies.

The agent and/or compound of methods and uses of the present invention is any physical or chemical agent, such as a pharmaceutical composition, cosmetic, drug, xenobiotic compound, food composition, sugar, lipid, protein, dietary supplement, radiation, and/or electrical stimuli. In a preferred embodiment, the compound is a sunlotion and/or said agent is UV-radiation. The agent and/or compound is for example in the form of solutions, crèmes, lotions, gels, microparticles, and/or nanoparticles, and the agent or compound is for example administered by oral, including buccal and sublingual, rectal, nasal, topical, pulmonary, vaginal, or parenteral, including intramuscular, intraarterial, intrathecal, subcutaneous and intravenous administration or administration by inhalation or insufflation; preferably the agent or compound is administered by topical and/or pulmonary administration.

In the methods of the present invention, the detection of the transcriptional and/or translational products is performed in the live animal; e.g. detection of the transcriptional and/or translational products is performed without removing the tissue from the live animal. In another embodiment, the detection of a transcriptional and/or translational reporter product is performed on a tissue sample removed from the animal. The tissue is for example selected from the group consisting of skin, epidermis, dermis, hypodermis, breast, fat, thymus, gut, small intestine, large intestine, stomach, muscle, pancreas, heart muscle, skeletal muscle, smooth muscle, liver, lung, brain, cornea and tumours, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and/or sweat; preferably tissue is skin, epidermis, and/or dermis.

In another aspect, the present invention relates to a cell line derived from a transgenic animal of the present invention.

Another aspect of the present invention relates to a transgenic non-human oocyte, sperm cell, blastocyst, embryo, fetus, donor cell, or cell nucleus derived from a transgenic non-human animal of the present invention, and/or a transgenic non-human oocyte, sperm cell, blastocyst, embryo, fetus, donor cell, or cell nucleus, wherein the transgenic genome comprises

i. at least one nucleic acid sequence encoding a nuclear receptor or part thereof and a DNA binding domain or part thereof, and

ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or

iii. the transcriptional or translational products of any of said nucleic acid sequences,

In yet another aspect, the present invention relates to a method of producing a transgenic non-human animal, oocyte, sperm cell, blastocyst, embryo, fetus, donor cell, or cell nucleus of the present invention comprising the steps of

i. providing a donor cell,

ii. genetically modifying the donor cell of i) by inserting

a. at least one nucleic acid sequence encoding a nuclear receptor or part thereof and a DNA binding domain or part thereof, and

b. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or

c. the transcriptional or translational products of any of said nucleic acid sequences,

iii. transferring the modified genome of the donor cell obtained in ii) into a host cell,

iv. obtaining a reconstructed embryo forming an embryo

v. culturing said embryo; and

vi. transferring said cultured embryo to a host mammal such that the embryo develops into a genetically modified fetus,

wherein said genetically modified embryo is produced by nuclear transfer comprises steps i) to v),

wherein said genetically modified blastocyst is produced by nuclear transfer comprises steps i) to vi),

wherein said genetically modified fetus is produced by nuclear transfer comprises steps i) to vi).

Moreover, an aspect of the present invention relates to use of a transgenic animal, a cell line, an oocyte, sperm cell, blastocyst, embryo, fetus, donor cell, and/or cell nucleus of the present invention for evaluating the activity of a nuclear receptor. In a preferred embodiment, the use relates to evaluating the effect of an agent on the activity of a nuclear receptor, for example an agent as defined above. In another embodiment, the use relates the evaluating in vivo the activity of a nuclear receptor due to endogenous agonists, for example due to agonists that are generated during normal development of the skin. In a specific embodiment, the endogenous agonists are generated during the development of a disease, such as psoriasis, different cancer types and/or other hyperproliferative diseases.

### DESCRIPTION OF DRAWINGS

FIG. 1. A: Principle of the nuclear receptor sensor system, C: The sleeping beauty (SB) genetic sensor.

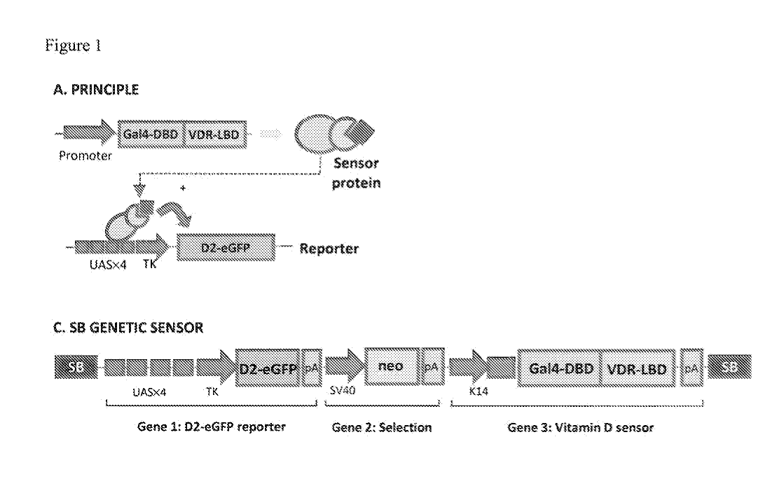

FIG. 2. A: pT2/UAS-d2eGFP; B: pT2/UAS-d2eGFP+pM/hVDR C: pT2/UAS-d2eGFP+Gal4VP16; D: pT2/UAS-d2eGFP+pM/hVDR+10̂-6 M alfacalcidol; E: pT2/UAS-d2eGFP+pM/hVDR+10̂-8 M alfacalcidol; F: pT2/UAS-d2eGFP+pM/hVDR+10̂-10 M alfacalcidol; G: pT2/UAS-d2eGFP+pM/hVDR+10̂-6 M calcipotriol; H: pT2/UAS-d2eGFP+pM/hVDR+10̂-8 M calcipotriol; I: pT2/UAS-d2eGFP+pM/hVDR+10̂-10 M calcipotriol

FIG. 3. Percentage of GFP-expressing cells as verified by FACS sorting of cells transfected with the nuclear receptor sensor vector constructs as indicated.

FIG. 4. Mean green fluorescence of GFP-expressing cells cells transfected with the nuclear receptor sensor vector constructs as indicated.

FIG. 5. A: Cis- construct with sensor and receptor component on the same plasmid. This construct is a preferred embodiment of the present invention for the production of transgenic pigs. B: Same construct as panel A, but with an SV40 promoter for expression of Gal4hVDR. C: Trans-acting constructs, wherein the sensor and receptor components are localized on separate plasmids. Trans-acting constructs may be used in situation where the functionality of cis-acting constructs are reduced. However, the use of trans-acting vectors holds the risk of losing one of the components in the breeding process. LIR: left inverted repeat; 4×UAS: 4 times upstream activating sequence; d2eGFP: destabilized enhanced green fluorescence protein; Neo: neomycin resistance gene; Gal4-hVDR: yeast transcription activator protein; RIR: right inverted repeat; miniTK promoter: minimal thymidin kinase promoter; SV40 promoter: simian virus 40 promoter; K14 promoter: human keratin 14 promoter med beta-globin intron.

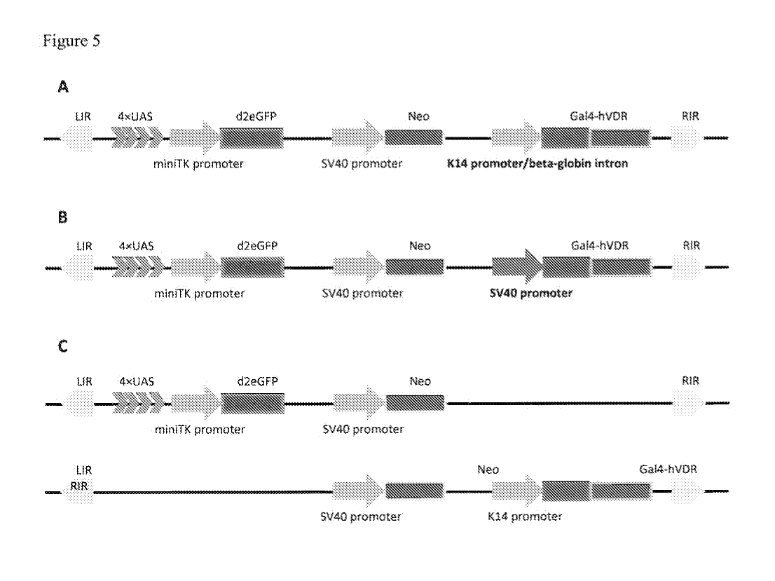

### DETAILED DESCRIPTION OF THE INVENTION

A methodology for detecting the activation of specific nuclear receptors is widely applicable. The present invention offers a method of detecting the activation of specific nuclear receptors. This methodology furthermore allows for the evaluation of the effect of a physical or chemical agent on the activation of specific nuclear receptors. In a further application, the method of the present invention also allows for the evaluation of the ability of a compound administered to a tissue before, after or in parallel with the administration of said physical or chemical agent to counteract or enhance the effect of said physical or chemical agent on the activation of nuclear receptors.

The invention allows detection of the activation of selected nuclear receptors in a tissue of an animal during all stages of development and/or after a previous administration of a compound that may alter the effect of said agent. The method of the present invention allows for the detection of the spatial and temporal activation of selected nuclear receptors.

The nuclear receptor sensor system according to the present invention can be used for several purposes, including:

i) studies of the effectiveness of pharmaceutical substances known to activate the selected nuclear receptors,

ii) determination of the activation of nuclear receptors due to the production of endogenous agonists during normal development and homeostasis of the tissue,

iii) studies of the penetration ability of various xenobiotics in the tissue, and

iv) studies of the ability of different types of liposomes, nanoparticles or other formulations to transport compounds into a tissue for drug delivery purposes. By treatment of the tissue with at test formula containing a known nuclear receptor activator, activation of the reporter system will reflect the penetration ability of the formulation.

Thus, in one aspect the present invention relates to a method for evaluating the effect of an agent in a tissue of an animal. This method comprises a) providing a transgenic animal, comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence, b) administering said agent to said animal, and c) evaluating the transcriptional and/or translational expression product of the nucleic acid sequence encoding the reporter polypeptide, wherein an alteration of said expression product prior to and after step (b) is indicative of an effect on said tissue.

In another aspect, the invention relates to a method for testing a compound for the ability to alter the effects of an agent in a tissue of an animal. This method comprises a) administering said compound to a transgenic animal comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence, b) administering said agent to said transgenic animal, and c) evaluating the transcriptional and/or translational expression product of the nucleic acid sequence encoding the reporter polypeptide, wherein a difference in the amount of said expression product in the presence and absence of said compound is indicative of said compound being able to alter the effect of said agent in said tissue.

In this context, the term “alter” comprises reducing or enhancing the effect of the physical or chemical agent on the nuclear receptor being evaluated. This aspect of the invention comprises administering said compound to the tissue of a transgenic animal, whose genome comprises a nucleic acid sequence encoding a reporter polypeptide, and an additional nucleic acid sequence encoding a nuclear receptor coupled to a DNA-binding domain, exposing the transgenic animal to a physical or chemical agent which is administered to a tissue, and measuring the expression of the nucleic acid sequence encoding the reporter polypeptide.

The term “transgenic animal” as used herein, refers to a non-human animal, which comprise a foreign gene in its genome. The foreign gene may be comprised in germ line tissue and thus, be transmitted to offspring. Exogenous genes can be transferred to the genome of the animal by techniques known to those of skill within the art. Preferred methods and techniques for production of transgenic animals are described elsewhere herein.

The term “in vivo” as used herein, refers to any process, reaction or experiment taking place within the body of a living animal.

The term “heterologous” as used herein refers to any combination of nucleic acid sequences that is not normally found intimately associated in nature. The heterologous genes according to the present invention are preferably selected from, but not limited to the group of reporter genes, nuclear receptors, promoters and enhancers as defines elsewhere herein.

The terms “evaluating”, “evaluation” or “evaluate” generally refers to the estimation of a parameter of interest. The estimation is typically based on a detection or determination of the parameter directly and/or an indicator of said parameter. Herein, specifically, the effect of an agent on the activity of a nuclear receptor is evaluated based on the detection of a reporter transcript or polypeptide.

The term “polynucleotide” or “nucleic acid sequence” refers to a polymeric form of nucleotides at least 2 bases in length. The term “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule. As used herein, the term “nucleic acid” includes DNAs or RNAs that contain one or more modified bases. Thus, DNAs or RNAs with backbones modified for stability or for other reasons are “ nucleic acid sequences” as that term is intended herein. Moreover, DNAs or RNAs comprising unusual bases, such as inosine, or modified bases, such as tritylated bases, to name just two examples, are nucleic acid sequences as the term is used herein. It will be appreciated that a great variety of modifications have been made to DNA and RNA that serve many useful purposes known to those of skill in the art. The term nucleic acid as it is employed herein embraces such chemically, enzymatically or metabolically modified forms of nucleic acid, as well as the chemical forms of DNA and RNA characteristic of viruses and cells, including simple and complex cells, inter alia.

A “fragment” or “part” thereof as used herein in relation to a nucleic acid sequence or a polypeptide is a unique portion of the nucleic acid sequence or polypeptide of the present invention, which is identical in sequence to but shorter in length than the parent sequence. Thus, the term ‘fragment’ or “part” refers to a nucleic acid sequence or polypeptide of the present invention, which may comprise up to the entire length of the defined sequence, minus one nucleotide or amino acid residues. For example, a fragment may comprise from 5 to 100000 contiguous nucleotides or amino acid residues. Fragments may be preferentially selected from certain regions of a molecule, for example a specific functional region, such as a ligand binding domain or a DNA-binding domain. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

The term “Homology” refers to sequence similarity or, interchangeably, sequence identity, between two or more polynucleotide sequences or two or more polypeptide sequences.

Methods of alignment of sequences for comparison are well-known in the art. Various programs and alignment algorithms are described and present a detailed consideration of sequence alignment methods and homology calculations, such as VECTOR NTI. The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences will be.

The NCBI Basic Local Alignment Search Tool (BLAST) is available from several sources, including the National Center for Biotechnology Information (NBCI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

Homologs of the disclosed polypeptides are typically characterised by possession of at least 94% sequence identity counted over the full length alignment with the disclosed amino acid sequence using the NCBI Basic Blast 2.0, gapped blastp with databases such as the nr or swissprot database. Alternatively, one may manually align the sequences and count the number of identical amino acids. This number divided by the total number of amino acids in your sequence multiplied by 100 results in the percent identity.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

The term “physically or chemically coupled” as used herein in respect of two or more polypeptide is meant to indicated that the polypeptides are linked by a physical and/or chemical interaction. Methods for chemical cross-linking by use of chemical or physical cross-linking agents are well-known in the art. In a preferred embodiment, physically or chemically coupled polypeptides of the present invention are expressed as a fusion peptide, wherein the polypeptides are coupled by a peptide bond (amide bond).

Fragment: is used to indicate a non-full length part of a nucleic acid or polypeptide. Thus, a fragment is itself also a nucleic acid or polypeptide, respectively. ??

Functional homologue: A functional homologue may be any nucleic acid/protein/polypeptide that exhibits at least some sequence identity with a wild type version/sequence of a given gene/gene product/protein/polypeptide and has retained at least one aspect of the original sequences functionality. Herein a functional homologue of HIV-1 envelope has the capability to induce an immune response to cells expressing HIV-1 envelope.

Promoter: A binding site in a DNA chain at which RNA polymerase binds to initiate transcription of messenger RNA by one or more nearby structural genes.

The terms “biological sample” or “sample” as used herein refers to any suitable biological sample comprising genetic material, such as RNA or DNA, and/or proteins. The sample is in a preferred embodiment, isolated from the subject, such as a pig, mouse, or another mammal. In a preferred embodiment the sample is a tissue sample selected from the group consisting of skin, epidermis, dermis, hypodermis, breast, fat, thymus, gut, small intestine, large intestine, stomach, muscle, pancreas, heart muscle, skeletal muscle, smooth muscle, liver, lung, brain, cornea and tumours, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and/or sweat. The most convenient sample type is a blood sample; however, the choice of sample depends on the specific disorder or clinical condition as well as detection method and will be evident for those of skill in the art.

The term “agonist” refers to a substance that mimics the function of an activating molecule. The term “antagonist” refers to a molecule that competes for the binding sites of an agonist, but does not induce an active response. Antagonists include, but are not limited to, drugs, hormones, antibodies, and neurotransmitters, as well as analogues and fragments thereof.

The term “ligand” refers to any molecule that binds to a specific site (ligand binding domain (LBD)) on another molecule. Thus, the term “ligand binding domain” is the site in e.g. a nuclear receptor, which binds a ligand. Binding of a ligand to a ligand binding domain of a polypeptide, such as a nuclear receptor induces conformational changes in the polypeptide, which may change the catalytic or regulatory activity of the polypeptide.

The term “modulate” encompasses an increase or a decrease, a stimulation, inhibition, or blockage in the measured activity when compared to a i suitable control. “Modulation” of expression levels includes increasing the level and decreasing the level of an mRNA or polypeptide encoded by a polynucleotide of the invention when compared to a control lacking the agent being tested. In some embodiments, agents of particular interest are those which inhibit a biological activity of a subject polypeptide, and/or which reduce a level of a subject polypeptide in a cell, and/or which reduce a level of a subject mRNA in a cell and/or which reduce the release of a subject polypeptide from a eukaryotic cell. In other embodiments, agents of interest are those that increase a biological activity of a subject polypeptide, and/or which increase a level of a subject polypeptide in a cell, and/or which increase a level of a subject mRNA in a cell and/or which increase the release of a subject polypeptide from a eukaryotic cell.

The term “gene product” as used herein refers to any transcriptional or translational product of a gene. A transcriptional product comprises any RNA-species, which is transcribed from the specific gene, such as pre-RNA, mRNA, tRNA, miRNA, spliced and nonspliced RNA. The transcript may be bound by RNA-binding proteins and, thus, packaged into a ribonucleoprotein (RNP), for example an mRNP molecule.

A translational gene product of the present invention comprises any peptide or polypeptide encoded by the gene or a fragment thereof. Thus, a “polypeptide encoded by a gene of the present invention” is comprised in the terms “gene product”, or “translational gene product”. A translational gene product of the present invention comprise any polypeptide-species encoded by a nucleic acid sequence of the present invention. For example, a translational gene product of the present invention comprises any polypeptide-species encoded by a sequence selected from any of SEQ ID NO: 1-??, or the complement thereof or part thereof.

The terms “increase” or “decrease” as used herein in respect of the expression of a transcriptional and/or translational gene product refers to a rise or reduction, respectively, of said transcriptional and/or translational gene product; i.e. the level of transcriptional and/or translational gene product is lower compared to the average level for example in an animal, tissue, and/or population of cells before or after a given treatment, e.g. administration of an agent or compound of the present invention. In respect of a transcriptional product of the present invention, the level of transcript may for example be determined by quantitative or semiquantitative reverse transcriptase polymerase chain reaction (RT-PCR). The level of transcript may be normalized according to an endogenous transcript. A decreased activity of a transcriptional product is for example observed by a reduction or decrease of the level of a specific RNA transcript, as determined for example by RT-PCR. In a preferred embodiment the level of RNA is determined by RT-PCR.

Decrease of the activity of a translational product comprises both a reduction in the amount/level of polypeptide, such as reporter polypeptide, and/or reduced enzymatic activity of said polypeptide and/or reduced ability of the polypeptide to interact with other polypeptides and signal cascades. The level of polypeptide may be determined by any suitable method available to those of skill in the art, for example by western blotting, or ELISA. The expression is in one embodiment increased by at least 10%, such as at least 20%, such at least 30%, such at least 40%, such at least 50%, such at least 60%, such at least 70%, such at least 80%, such at least 90%, such at least 100%, such at least 200%, such at least 300%, such at least 400%, such at least 500%, such at least 600%, such at least 700%, such at least 800%, such at least 900%, such at least 1000% in the presence of an agent of the present invention compared with the absence of said agent. In another embodiment, the expression is decreased to less than 95%, such as less than 90%, such as less than 80%, such as less than 70%, such as less than 60%, such as less than 50%, such as less than 40%, such as less than 30%, such as less than 20%, such as less than 10%, such as less than 9%, such as less than 8%, such as less than 7%, such as less than 6%, such as less than 5%, such as less than 4%, such as less than 3%, such as less than 2%, such as less than 1%, such as less than 0.5% in the presence of an agent of the present invention compared with the absence of said agent.

The Nuclear Receptor Sensor System

The method of the present invention comprises a molecular sensor system for detection of nuclear receptor activation in vivo. The nuclear receptor system comprises a sensor component and a reporter component. The nuclear receptor sensor system relies on molecular interaction between the sensor system and the reporter system within the cells of a tissue. The sensor system comprises a nuclear receptor or a fragment thereof, which is physically or chemically coupled, e.g. fused, to a heterologous DNA binding domain. Ligand binding to the nuclear receptor induces a conformational change of the fusion polypeptide, which associates with the DNA element specific for the DNA binding domain of the fusion polypeptide, thereby promoting transcription of downstream gene clusters.

The present invention also relates to a non-human transgenic animal, which comprises a nuclear receptor sensor cassette and/or a reporter cassette, wherein the sensor cassette generally comprises at least one nucleic acid sequence encoding a nuclear receptor or part thereof and a DNA binding domain or part thereof, and the sensor cassette generally comprises at least one nucleic acid sequence encoding a detectable reporter nucleic acid transcript and/or reporter polypeptide or part thereof, wherein said nucleic acid further comprise at least one binding site for a polypeptide comprising a DNA binding domain.

In a preferred embodiment to the non-human transgenic animal, preferably a pig, as well as an oocyte, sperm cell, blastocyst, embryo, fetus, donor cell, or cell nucleus as well as methods and uses of the present invention, the expression of the nuclear receptor or part thereof and a DNA binding domain or part thereof promotes expression of said reporter polypeptide. In particular, when a ligand or an agonist for said nuclear receptor is present, the nuclear receptor or part thereof, such as the ligand binding domain of said nuclear receptor promotes the expression of reporter transcript or polypeptide.

Sensor System

The present invention relates to a non-human transgenic animal, preferably a pig, as well as an oocyte, sperm cell, blastocyst, embryo, fetus, donor cell, or cell nucleus which comprises in its genome a sensor component and/or a reporter component. Generally, the sensor component comprises at least one nucleic acid sequence encoding a nuclear receptor or part thereof and a DNA binding domain or part thereof, and/or the transcriptional or translational products of any of said nucleic acid sequences.

Thus, the present invention comprises one nucleic acid sequence encoding a reporter polypeptide and an additional nucleic acid sequence encoding a fusion polypeptide, comprising a nuclear receptor or part thereof coupled to a DNA binding domain, or the transcriptional or translational products of said additional nucleic acid sequence. That additional nucleic acid sequence is part of the sensor system of the present invention. In one embodiment, the sensor system according to the present invention comprises a nucleic acid cassette encoding a fusion peptide comprising a DNA binding domain and a ligand binding domain of a nuclear receptor.

In one embodiment, the nucleic acid sequence or additional nucleic acid sequence encoding the sensor system is preceded by a promoter. Thus, the said nuclear receptor or part thereof and a DNA binding domain or part thereof is in one embodiment expressed from an inducible promoter and/or a tissue-specific promoter, or an inducible tissue-specific promoter. In a specific embodiment, the promoter is an inducible promoter. In one embodiment, the nucleic acid sequence or additional nucleic acid sequence comprises a tissue specific enhancer/promoter to target the expression of the fusion peptide to a specific tissue. Thereby, the reporter systems of the present invention can be activated by expressing such fusion peptides. Specifically, the tissue-specific promoter is specific for a tissue selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and/or tumours. In a preferred embodiment, the promoter is a skin-specific promoter. In another preferred embodiment, the promoter is keratin 14 enhancer/promoter.

Thus, in one such embodiment, the additional nucleic acid sequence encoding a nuclear receptor coupled to a DNA binding domain is expressed from a tissue-specific promoter. In one embodiment, the promoter comprises enhancer elements. In one embodiment, the promoter comprises a light-inducible sequence. In yet another embodiment, the promoter comprises a chemically inducible sequence.

The DNA-binding domain of the sensor component is any polypeptide or other chemical group, which may be coupled to the nuclear receptor or part thereof of the sensor component. The function of the DNA-binding domain is to direct the nuclear receptor or part thereof to a target region, for example the promoter region or upstream activation sequence of the reporter gene of the present invention. In a preferred embodiment, the DNA-binding domain is the yeast (Saccharomyces cerevisiae) Gal4 upstream activation region (GAL 4 UAS or UAS_{gal}), or any part or functional homolog thereof. In another preferred embodiment, the DNA-binding domain is the bacterial LexA DNA-binding domain, or any part or functional homolog thereof. In another embodiment, the DNA-binding domain is yeast UAS_{gal}, LexA DNA-binding domain, or any part or functional homolog thereof.

In a preferred embodiment, the nuclear receptor or part thereof and a DNA binding domain or part thereof are physically or chemically coupled.

Several methods for chemical cross-linking by use of chemical or physical cross-linking agents are available to the person of skill in the art. In a preferred embodiment, the nuclear receptor or part thereof and DNA binding domain or part thereof are expressed as a fusion peptide, wherein the polypeptides are coupled by a peptide bond (amide bond). Thus, in a preferred embodiment, the sensor polypeptides are expressed as a fusion peptide.

In a preferred embodiment, the DNA binding domain of the sensor component is selected from the group consisting of yeast GAL4 DNA binding domain and/or the LexA DNA binding domain, and the ligand binding domains are derived from a nuclear receptor, such as the retinoic acid receptor, the vitamin D receptor, the liver X receptors, the promiscuous pregnane X receptor or the PPARs. In a specific embodiment, the promoter regions of the fusion construct is replaced by the keratin 14 enhancer/promoter, a promoter known to drive epidermis specific expression in order to ensure skin-specific expression.

In a preferred embodiment, the nuclear receptor or part thereof comprised in the fusion polypeptide of the present invention comprise at least one ligand binding domain or a fragment of a ligand binding domain of a nuclear receptor as defined herein below.

The nuclear receptor or part thereof may be inserted anywhere in the DNA binding domain and/or be coupled to any of the terminals of DNA-binding domains. Thus, in one embodiment, the fusion polypeptide comprises a nuclear receptor or part thereof inserted within, and/or at the N-terminus and/or C-terminus of a DNA binding domain or part thereof. In a specific embodiment, the fusion polypeptide comprises a nuclear receptor or part thereof inserted at the C-terminus of a DNA binding domain or part thereof.

In a preferred embodiment of the methods, animals and/or cells of the present invention expression of the fusion polypeptide of this invention promotes expression of any reporter polypeptide, as defined herein.

Nuclear Receptors

The present invention allows detection of the activation of selected nuclear receptors in a tissue. Detection can take place at all stages of development of that tissue. Nuclear receptors belong to a class of proteins that are responsive to hormones and certain other molecules. Nuclear receptors work in concert with other proteins to increase the expression of specific genes.

Nuclear receptors are classified as transcription factors: they interact with DNA and regulate the expression of adjacent genes. This regulation of gene expression by nuclear receptors is ligand dependent, and nuclear receptors are normally only active in the presence of a ligand. A ligand is a chemical substance, of which the binding to a nuclear receptor results in a conformational change in the receptor resulting in the activation of the receptor and up-regulation of the expression of the corresponding gene. Ligands that bind activate nuclear receptors include lipophilic substances such as endogenous hormones, vitamins A and D, and xenobiotics.

Expression of a large number of genes is regulated by nuclear receptors and, ligands that activate these receptors may have severe effects on the organism. In particular, nuclear receptors regulate genes, which are associated with various disorders, such as multiple cancer types and other hyperproliferative disorders. Consequently, nuclear receptors are common targets of a wide range of pharmaceuticals. Moreover, nuclear receptors play an important role in the tempero-spatial regulation of gene expression during development and homeostasis of organisms.

The specific group of nuclear receptors called orphan receptors have no known endogenous ligands. Some of these receptors, for example FXR, LXR, and PPAR bind a number of metabolic intermediates such as fatty acids, bile acids and/or sterols with relatively low affinity, and may thereby function as metabolic sensors. Other nuclear receptors, such as CAR and PXR appear to function as xenobiotic sensors that stimulate expression of cytochrome P450 enzymes that metabolize these xenobiotics.

Nuclear receptors have a modular structure and contain the following domains A-F:

A-B) N-terminal regulatory domain: Contains the activation function 1 (AF-1), which is ligand independent. The transcriptional activation effect of AF-1 is normally very weak, but in combination with activation function 2 (AF-2), mentioned below, it contributes to produce a more robust upregulation of gene expression. The A-B domain is highly variable in sequence.

C) DNA binding domain (DBD): This domain containing two zinc fingers, which bind to specific DNA regions called hormone response elements (HRE). DBD is highly conserved.

D) Hinge region: This flexible domain connects the DBD with the LBD, see below. The hinge region also influences intracellular trafficking and subcellular distribution.

E) Ligand binding domain (LBD): The LBD is structured as an alpha helical sandwich fold in which three anti parallel alpha helices are flanked by two alpha helices on one side and three on the other. The ligand binding cavity is located within the interior of the LBD and just below the three anti parallel alpha helices. Along with the DBD, the LBD contributes to dimerization of the receptor as well as binding of coactivator and corepressor proteins. This domain also comprises the activation function 2 (AF-2), the action of which is dependent on the ligand binding. LBD is moderately conserved in sequence and highly conserved in structure between different nuclear receptors.

F) C-terminal domain: This domain varies in sequence between various nuclear receptors.

In the general mechanism, the binding of a ligand to the nuclear receptor leads to a conformational change of the receptor, which triggers a number of down stream events that eventually results in up or down regulation of gene expression. According to their specific mechanism of action and subcellular distribution in the absence of ligand, nuclear receptors can be classified into two broad classes. Type I nuclear receptors are predominantly located in the cytosol, while type II nuclear receptors are located in the nucleus.

Type I

The binding of ligand to type I nuclear receptors in the cytosol results in the dissociation of heat shock proteins and homodimerization, followed by translocation from the cytoplasm into the cell nucleus, where the nuclear receptor binds to specific DNA regions known as hormone response elements (HREs). Type I nuclear receptors bind to HREs consisting of two half sites separated by a variable length of DNA, wherein the second half site is an inverted repeat of the first. The nuclear receptor/DNA complex then induces the recruitment of other proteins, which ultimately activates transcription of genes located downstream of the HRE.

Type II

Type II receptors are predominantly retained in the nucleus regardless of ligand binding status. Additionally, type II nuclear receptors bind to DNA as heterodimers, usually with RXR. In the absence of ligand, type II nuclear receptors are often complexed with corepressor proteins. Ligand binding to the nuclear receptor results in dissociation of corepressor and recruitment of coactivator proteins. Additional proteins including RNA polymerase are then recruited to the nuclear receptor/DNA complex to activate transcription of downstream genes.

Type III

Type III nuclear receptors (principally NR subfamily 2) are similar to type I receptors in binding to DNA has homodimers. However, type III bind to direct repeat instead of inverted repeat HREs.

Type IV

Type IV nuclear receptors may bind both as monomers or dimmers. However, only a single DNA binding domain of the nuclear receptor binds to a single half site HRE. Examples of type IV receptors are found in most of the NR subfamilies.

Agonism and Antagonism

Depending on the receptor involved, the chemical structure of the ligand and the tissue that is being affected, nuclear receptor ligands may display dramatically diverse effects ranging from agonism to antagonism and inverse agonism.

Agonists

The binding of ligands to their cognate nuclear receptors may lead to upregulation of gene expression. This stimulation of gene expression by the ligand is referred to as an agonist response. The agonistic effects of endogenous hormone ligands can also be mimicked by certain synthetic ligands, for example the glucocorticoid receptor anti-inflammatory drug dexamethasone. Agonist ligands work by inducing a conformation of the receptor which favors coactivator binding. Coactivators are recruited by the nuclear receptor upon binding to the DNA, and serves to activate transcription. Coactivators often have an intrinsic histone acetyltransferase (HAT) activity, which weakens the association of histones to DNA, and thereby promotes gene transcription.

Antagonists

Some synthetic nuclear receptor ligands have no apparent effect on gene transcription in the absence of endogenous ligand. However, they can block the effect of agonist ligand through competitive binding to the same site of the nuclear receptor. Such ligands are known as antagonists. Antagonists are commonly used as pharmaceuticals such as the antagonistic nuclear receptor drug is mifepristone, which binds to the glucocorticoid and progesterone receptors, thereby blocking the activity of the endogenous hormones cortisol and progesterone respectively. Antagonist ligands work by inducing a conformation of the receptor which prevents coactivator binding and promotes corepressor association. Corepressors often work by recruiting histone deacetylases (HDACs), which strengthens the association of histones to DNA, and thus represses gene transcription.

Inverse Agonists

Some nuclear receptors are constitutively active, stimulating DNA transcription in the absence of agonists. This constitutive activity can be repressed by synthetic ligands, known as inverse agonists.

Selective Receptor Modulators

Some pharmaceutical compounds directed towards nuclear receptors display an agonist response in some tissue and an antagonistic response in other tissues. This behavior allow the retaining of a desired beneficial therapeutic effect of a drug in one tissue, while minimizing undesirable side effects of the drug in other tissues. Drugs with this mixed agonist/antagonist behavior are referred to as selective receptor modulators (SRMs). Examples include Selective Estrogen Receptor Modulators (SERMs) and Selective Progesterone Receptor Modulators (SPRMs). The mechanism of action of SRMs varies depending on the chemical structure of the ligand and the receptor involved. It is, however, generally believed that many SRMs work by promoting a conformation of the receptor that is closely balanced between agonism and antagonism. In tissues where the concentration of coactivator proteins is higher than corepressors, the equilibrium is shifted in the agonist direction, and conversely in tissues where corepressors dominate, the ligand behaves as an antagonist.

Family Members

Below is a list of 48 known human nuclear receptors categorized according to sequence homology. The nuclear receptors are organized by

Subfamily: name
Group: name (endogenous ligand if common to entire group)
   Member: name (abbreviation; NRNC Symbol, gene) (endogenous ligand)

Subfamily 1: Thyroid Hormone Receptor-Like
Group A: Thyroid hormone receptor (Thyroid hormone)
   1: Thyroid hormone receptor-α (TRα; NR1A1, THRA)
   2: Thyroid hormone receptor-β (TRβ; NR1A2, THRB)
Group B: Retinoic acid receptor (Vitamin A and related compounds)
   1: Retinoic acid receptor-α (RARα; NR1B1, RARA)
   2: Retinoic acid receptor-β (RARβ; NR1B2, RARB)
   3: Retinoic acid receptor-γ (RARγ; NR1B3, RARG)
Group C: Peroxisome proliferator-activated receptor
   1: Peroxisome proliferator-activated receptor-α (PPARα; NR1C1, PPARA)
   2: Peroxisome proliferator-activated receptor-β/δ (PPARβ/δ; NR1C2, PPARD)
   3: Peroxisome proliferator-activated receptor-γ (PPARγ; NR1C3, PPARG)
Group D: Rev-ErbA
   1: Rev-ErbAα (Rev-ErbAα; NR1D1)
   2: Rev-ErbAβ (Rev-ErbAβ; NR1D2)
Group F: RAR-related orphan receptor
   1: RAR-related orphan receptor-α (RORα; NR1F1, RORA)
   2: RAR-related orphan receptor-β (RORβ; NR1F2, RORB)
   3: RAR-related orphan receptor-γ (RORγ; NR1F3, RORC)
Group H: Liver X receptor-like
   3: Liver X receptor-α (LXRα; NR1H3)
   2: Liver X receptor-β (LXRβ; NR1H2)
   4: Farnesoid X receptor (FXR; NR1H4)
Group I: Vitamin D receptor-like
   1: Vitamin D receptor (VDR; NR1I1, VDR) (vitamin D)
   2: Pregnane X receptor (PXR; NR1I2)
   3: Constitutive androstane receptor (CAR; NR1I3)

Subfamily 2: Retinoid X Receptor-Like
Group A: Hepatocyte nuclear factor-4 (HNF4)
   1: Hepatocyte nuclear factor-4-α (HNF4α; NR2A1, HNF4A)
   2: Hepatocyte nuclear factor-4-γ (HNF4γ; NR2A2, HNF4G)
Group B: Retinoid X receptor (RXRα)
   1: Retinoid X receptor-α (RXRα; NR2B1, RXRA)
   2: Retinoid X receptor-β (RXRβ; NR2B2, RXRB)
   3: Retinoid X receptor-γ (RXRγ; NR2B3, RXRG)
Group C: Testicular receptor
   1: Testicular receptor 2 (TR2; NR2C1)
   2: Testicular receptor 4 (TR4; NR2C2)
Group E: TLX/PNR
   1: Human homologue of the Drosophila tailless gene (TLX; NR2E1)
   3: Photoreceptor cell-specific nuclear receptor (PNR; NR2E3)
Group F: COUP/EAR
   1: Chicken ovalbumin upstream promoter-transcription factor I (COUP-TFI; NR2F1)
   2: Chicken ovalbumin upstream promoter-transcription factor II (COUP-TFII; NR2F2)
   6: V-erbA-related (EAR-2; NR2F6)

Subfamily 3: Estrogen Receptor-Like
Group A: Estrogen receptor (Sex hormones: Estrogen)
   1: Estrogen receptor-α (ERα; NR3A1, ESR1)
   2: Estrogen receptor-β (ERβ; NR3A2, ESR2)
Group B: Estrogen related receptor
   1: Estrogen related receptor-α (ERRα; NR3B1, ESRRA)
   2: Estrogen related receptor-β (ERRβ; NR3B2, ESRRB)
   3: Estrogen related receptor-γ (ERRγ; NR3B3, ESRRG)
Group C: 3-Ketosteroid receptors
   1: Glucocorticoid receptor (GR; NR3C1) (Cortisol)
   2: Mineralocorticoid receptor (MR; NR3C2) (Aldosterone)
   3: Progesterone receptor (PR; NR3C3, PGR) (Sex hormones: Progesterone)
   4: Androgen receptor (AR; NR3C4, AR) (Sex hormones: Testosterone)

Subfamily 4: Nerve Growth Factor IB-Like
Group A: NGFIB/NURR1/NOR1
   1: Nerve Growth factor IB (NGFIB; NR4A1)
   2: Nuclear receptor related 1 (NURR1; NR4A2)
   3: Neuron-derived orphan receptor 1 (NOR1; NR4A3)

Subfamily 5: Steroidogenic Factor-Like
Group A: SF1/LRH1
   1: Steroidogenic factor 1 (SF1; NR5A1)
   2: Liver receptor homolog-1 (LRH-1; NR5A2)

Subfamily 6: Germ Cell Nuclear Factor-Like
Group A: GCNF
   1: Germ cell nuclear factor (GCNF; NR6A1)

Subfamily 0: Miscellaneous
Group B: DAX/SHP
   1: DAX1, Dosage-sensitive sex reversal, adrenal hypoplasia critical region, on chromosome X, gene 1 (NR0B1)
   2: Small heterodimer partner (SHP; NR0B2)
Group C: Nuclear receptors with two DNA binding domains (2DBD-NR) (A novel subfamily)

The present invention offers a transgenic animal, and cells derived therefrom for detecting the activity of selected nuclear receptors, as well as methods of detecting the activation or activity of specific nuclear receptors. Specifically, the present invention provides a transgenic animal, preferably a pig, as well as an oocyte, sperm cell, blastocyst, embryo, fetus, donor cell, or cell nucleus, which comprises at least one nucleic acid sequence encoding a nuclear receptor or part thereof and a DNA binding domain or part thereof, and/or the transcriptional or translational products of any of said nucleic acid sequences. The nuclear receptor according to the present invention is any mammalian or non-mammalian nuclear receptor, including any of the nuclear receptors listed above.

In one embodiment of the present invention, the nuclear receptor is selected from the group consisting of Thyroid hormone receptor-α (TRα; NR1A1, THRA), Thyroid hormone receptor-β (TRβ; NR1A2, THRB), Retinoic acid receptor-α (RARα; NR1B1, RARA), Retinoic acid receptor-β (RARβ; NR1B2, RARB), Retinoic acid receptor-γ (RARγ; NR1B3, RARG), Peroxisome proliferator-activated receptor-α (PPARα; NR1C1, PPARA), Peroxisome proliferator-activated receptor-β/δ (PPARβ/δ; NR1C2, PPARD), Peroxisome proliferator-activated receptor-γ (PPARγ; NR1C3, PPARG), Rev-ErbAα (Rev-ErbAα; NR1D1), Rev-ErbAβ (Rev-ErbAβ; NR1D2), RAR-related orphan receptor-α (RORα; NR1F1, RORA), RAR-related orphan receptor-β (RORβ; NR1F2, RORB), Liver X receptor-α (LXRα; NR1H3), Liver X receptor-β (LXRβ; NR1H2), Farnesoid X receptor (FXR; NR1H4), Vitamin D receptor (VDR; NR1I1, VDR) (vitamin D), Pregnane X receptor (PXR; NR1I2), Constitutive androstane receptor (CAR; NR1I3), Hepatocyte nuclear factor-4-α (HNF4α; NR2A1, HNF4A), Hepatocyte nuclear factor-4-γ (HNF4γ; NR2A2, HNF4G), Retinoid X receptor-α (RXRα; NR2B1, RXRA), Retinoid X receptor-β (RXRβ; NR2B2, RXRB), Retinoid X receptor-γ (RXRγ; NR2B3, RXRG), Testicular receptor 2 (TR2; NR2C1), Testicular receptor 4 (TR4; NR2C2), Human homologue of the Drosophila tailless gene (TLX; NR2E1), Photoreceptor cell-specific nuclear receptor (PNR; NR2E3), Chicken ovalbumin upstream promoter-transcription factor I (COUP-TFI; NR2F1), Chicken ovalbumin upstream promoter-transcription factor II (COUP-TFII; NR2F2), 6: V-erbA-related (EAR-2; NR2F6), Estrogen receptor-α (ERα; NR3A1, ESR1), Estrogen receptor-β (ERβ; NR3A2, ESR2), Estrogen related receptor-α (ERRα; NR3B1, ESRRA), Estrogen related receptor-β (ERRβ; NR3B2, ESRRB), Estrogen related receptor-γ (ERRγ; NR3B3, ESRRG), Glucocorticoid receptor (GR; NR3C1) (Cortisol), Mineralocorticoid receptor (MR; NR3C2) (Aldosterone), Progesterone receptor (PR; NR3C3, PGR) (Sex hormones: Progesterone), Androgen receptor (AR; NR3C4, AR) (Sex hormones: Testosterone), Nerve Growth factor IB (NGFIB; NR4A1), Nuclear receptor related 1 (NURR1; NR4A2), Neuron-derived orphan receptor 1 (NOR1; NR4A3), Steroidogenic factor 1 (SF1; NR5A1), Liver receptor homolog-1 (LRH-1; NR5A2), Germ cell nuclear factor (GCNF; NR6A1), DAX1 (Dosage-sensitive sex reversal, adrenal hypoplasia critical region, on chromosome X, gene 1 (NR0B1)), Small heterodimer partner (SHP; NR0B2) and/or Nuclear receptors with two DNA binding domains (2DBD-NR). Each of the nuclear receptors specified above is intended to be an individual embodiment. Consequently, detection of the activation of each of them according to the present invention may be claimed individually.

In one preferred embodiment, the nuclear receptor is selected from the group consisting of vitamin D receptor, Liver X receptors, Retinoic Acid receptor, Retinoid X receptor, promiscuous pregnane X receptor and/or peroxisome proliferation activation receptors (PPARs), including PPARα, PPARβ/δ, PPARγ. In one preferred embodiment, the nuclear receptor is selected from the group consisting of Peroxisome proliferator-activated receptors (PPARs). In a specifically preferred embodiment, the nuclear receptor is PPARβ/δ. In another specifically preferred embodiment, the nuclear receptor is PPARα. In another specifically preferred embodiment, the nuclear receptor is PPARβ. In another specifically preferred embodiment, the nuclear receptor is PPARγ. In another specifically preferred embodiment, the nuclear receptor is PPARδ. PPARδ is the main PPAR subtype expressed in human epidermis. In another preferred embodiment, the nuclear receptor is Pregnane X receptor (PXR). PXR is a likely target for numerous xenobiotics. In another preferred embodiment, the nuclear receptor is selected from the retinoic acid receptors (RARs). The retinoic acid receptor is a validated skin target and regulator of skin homeostasis. In a specifically preferred embodiment, the nuclear receptor is Retinoic acid receptor-α (RARα). In another specifically preferred embodiment, the nuclear receptor is Retinoic acid receptor-β (RARβ). In an even further specifically preferred embodiment, the nuclear receptor is Retinoic acid receptor-γ (RARγ). In a further preferred embodiment, the nuclear receptor is Vitamin D receptor. Vitamin D receptor is a known pharmaceutical target in the treatment of psoriasis. Any part or functional homolog of any one of the nuclear receptors mentioned herein is also within the scope of the present invention.

In a preferred embodiment, the nuclear receptor or part thereof is the ligand-binding domain of a nuclear receptor or part thereof, such as listed above.

In a most preferred embodiment, the non-human transgenic animal, preferably a pig, as well as an oocyte, sperm cell, blastocyst, embryo, fetus, donor cell, or cell nucleus of the present invention comprises at least one nucleic acid sequence encoding a fusion polypeptide, comprising PPARδ or part thereof coupled to yeast GAL4 DNA binding domain and/or at least one nucleic acid sequence encoding β-galactosidase or part thereof.

Reporter System

The present invention relates to a non-human transgenic animal, preferably a pig, as well as an oocyte, sperm cell, blastocyst, embryo, fetus, donor cell, or cell nucleus which comprises in its genome a sensor component and/or a reporter component. Generally, the reporter component comprises at least one nucleic acid sequence encoding a detectable reporter nucleic acid transcript and/or reporter polypeptide or part thereof, wherein said nucleic acid further comprise at least one binding site for a polypeptide comprising a DNA binding domain and/or the transcriptional or translational products of any of said nucleic acid sequences.

Thus, the present invention comprises one nucleic acid sequence encoding a reporter transcript or polypeptide. The reporter system comprises a cassette comprising a nucleic acid sequence encoding a reporter polypeptide. The reporter system may be comprised in a vector as described elsewhere herein. The term “reporter gene” as used herein refers to any gene, of which a transcriptional activation can be detected. Thus, the methods, animals and cells of the present invention in one embodiment comprise a reporter polypeptide or fragment thereof comprising a detectable product. Detection of transcriptional activation is described elsewhere herein, however; in general the reporter polypeptide or fragment thereof comprises a visually, optically or autoradiographically detectable product.

The animal, preferably a pig, as well as an oocyte, sperm cell, blastocyst, embryo, fetus, donor cell, or cell nucleus comprise a nucleic acid encoding a detectable reporter nucleic acid transcript and/or reporter polypeptide or part thereof. A number of reporter genes and systems for detection exist which will be appreciated by a person skilled in the art. For example the reporter gene or nucleic acid encoding a reporter polepeptide according to the present invention is selected from the group consisting of β-galactosidase, HcRed, DsRed, DsRed monomer, ZsGreen, AmCyan, ZsYellow, fire fly luciferase, lac Z, renilla luciferase, SEAP, enhanced green fluorescent protein (eGFP), d2EGFP, enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, human alpha-1-antitrypsin (hAAT) and/or fragments, modifications and/or functional variants thereof.

It is understood that any of β-galactosidase, HcRed, DsRed, DsRed monomer, ZsGreen, AmCyan, ZsYellow, fire fly luciferase, lac Z, renilla luciferase, SEAP, enhanced green fluorescent protein (eGFP), d2EGFP, enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, human alpha-1-antitrypsin (hAAT) and/or fragments, modifications or functional variants thereof may be used in each their separate embodiment. In a preferred embodiment, the reporter gene or nucleic acid encoding a reporter polepeptide is β-galactosidase, or a variant or functional homolog thereof. In another preferred embodiment, the reporter gene is eGFP, or a variant or functional homolog thereof.

In a preferred embodiment of the present invention, the reporter gene or nucleic acid encoding a reporter polepeptide is the β-galactosidase gene, or a functional homolog or part thereof. This enzyme is encoded by the lacZ gene in the lac operon of Escherichia coli, and splits lactose into glucose and galactose. β-galactosidase is also produced in humans in the digestive tract. Use of β-galactosidase as the reporter gene allows for simple enzymatic detection of expression by methods known to those of skill within the art. Methods of detection and analysis according to the present invention are described elsewhere herein.

In another preferred embodiment of the present invention, the reporter gene is a fluorescent protein. In a specifically preferred embodiment, the reporter gene is green fluorescent protein, or a derivative or functional homolog thereof, including enhanced green fluorescent protein, yellow fluorescent protein and red fluorescent protein. The use of reporter genes encoding fluorescent peptides allows for direct fluorescent detection by confocal and multiphoton fluorescent microscopy.

The cassette comprising a nucleic acid sequence encoding a reporter polypeptide may further comprise promoter elements. In one embodiment, the nucleic acid sequence encoding a reporter polypeptide is preceded by a promoter. In a specific embodiment, the cassette comprises a promoter that drives the expression of a reporter gene. In one embodiment, the promoter is a heterologous promoter. In another embodiment, the promoter is an inducible promoter. In a specific embodiment, the promoter is thymidin kinase promoter, or a fragment or functional homolog thereof. In a preferred embodiment, the promoter element is thymidin kinase promoter.

The cassette comprising a nucleic acid sequence encoding a reporter polypeptide may further comprise a least one enhancer and/or regulatory element. An enhancer element is a regulatory DNA sequence that promotes the transcription of a gene. Enhancers may increase the rate of genetic transcription by increasing the activity of the nearest promoter on the same DNA molecule. An enhancer does not need to be particularly close to the genes it acts on, but enhancers are predominantly located on the same nucleic acid sequence as the genes that it acts on, although exceptions occur.

In one embodiment, the nucleic acid sequence encoding a reporter polypeptide is preceded by an enhancer. In a specific embodiment, the cassette comprises at least one enhancer element that promotes expression of a reporter gene. In one embodiment, the at least one enhancer element is a heterologous enhancer. In a specific embodiment, the enhancer element is selected from the group consisting of the yeast UAS_{gal} enhancer and/or the bacterial LexA binding site. In a preferred embodiment, the enhancer element is yeast UAS_{gal} enhancer, or a fragment or functional homolog thereof. In another preferred embodiment, the enhancer element is the bacterial LexA binding site, or a fragment or functional homolog thereof. The enhancer/promoter is preferably conventional combinations of the yeast UAS_{gal} enhancer or the bacterial LexA binding site fused with the thymidin kinase promoter.

In a preferred embodiment of the transgenic animal, oocyte, sperm cell, blastocyst, embryo, fetus, donor cell, or cell nucleus, uses and methods of the present invention, the nucleic acid sequence encoding a detectable reporter nucleic acid transcript and/or reporter polypeptide or part thereof further comprises at least one yeast Gal4 upstream activation sequence (UASgal), bacterial LexA binding site and/or a part thereof. Moreover, the nucleic acid sequence encoding a detectable reporter transcript or polypeptide is expressed from a heterologous and/or inducible promoter, such as defined above.

Analysis and Detection

The present invention offers a method for evaluating the effect of an agent or compound in a tissue of an animal, wherein an alteration of expression product prior to and after step administration of said agent is indicative of an effect on said tissue. An agent or compound is regarded to have an effect on a tissue, if the amount of expression product is increased or decreased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 225%, at least 250%, at least 275%, at least 300%, at least 350%, at least 400%, at least 450%, at least 500%, at least 550%, at least 600%, at least 750%, at least 800%, at least 850%, at least 900%, at least 950%, at least 1000%, at least 1100%, at least 1200%, at least 1300%, at least 1400%, at least 1500%, at least 1600%, at least 1700%, at least 1800%, at least 1900%, at least 2000%, at least 2500%, at least 3000%, at least 3500%, at least 4000%, at least 4000%, at least 4500%, at least 5000%, at least 5500%, at least 6000%, at least 6500%, at least 6500%, at least 7000%, at least 7500%, at least 8000%, at least 8500%, at least 9000%, at least 10000%, at least 15000%, at least or 20000%.

The term “evaluation” is used herein to comprise detection of an activation of the reporter gene according to the present invention. Detection may be achieved by measuring the level of transcriptional or translational product, i.e. the expression product comprise RNA and/or polypeptide.

In general, the reporter transcript, polypeptide or fragment thereof of the animal, oocyte, sperm cell, blastocyst, embryo, fetus, donor cell, or cell nucleus present invention comprises a visually, optically or autoradiographically detectable product. Thus, in one embodiment the methods, animals, embryos, blastocysts and/or cells of the present invention comprises a reporter polypeptide or fragment thereof comprising a detectable product. The reporter polypeptide or fragment or functional homolog thereof comprises a visually, optically and/or autoradiographically detectable product. However, detection may be performed by any technique known to people of skill within the art, including enzymatic and spectroscopic assays, confocal and multiphoton fluorescent microscopy, western blotting, imunostaining, Enzyme-linked immunosorbent assay (ELISA) as well as nucleic acid detection techniques such as northern blotting, southern blotting, polymerase chain reaction, primer extension and/or DNA array technologies. Moreover, specific PCR based techniques such as RT-PCR, q-PCR, as well as fluorescence microscopy and immunohistochemiestry may be employed.

When using β-galactosidase as the reporter gene according to the present invention, the expression product can be detection by use of enzymatic methods known to those of skill within the art. Several methods and commercial kits exist for fast and convenient detection and quantification of β-galactosidase activity. In one embodiment, the β-galactosidase activity is measured by providing a substrate, such as X-Gal (5-bromo-4-chloro-3-indolyl-beta-D-galactopyranoside). X-gal is an inert chromogenic substrate for β-galactosidase. β-galactosidase hydrolyzes X-Gal into a colorless galactose and 4-chloro-3-brom-indigo which forms an intense blue precipitate, which can be detected by optical and/or microscopic methods. However β-galactosidase expression may also be detected by anti-β-galactosidase antibodies, by use of immunoassays, such as ELISA, western blotting, and in situ hybridization.

The evaluation may be performed on a sample removed from the animal. In one embodiment, the evaluation is performed on samples selected from the group consisting of breast tissue, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and sweat.

In preferred embodiments the sample is selected from the group consisting of skin tissue, including epidermal and dermal tissue, breast tissue, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and sweat. In another embodiment the sample is selected from the group consisting of breast tissue, ovarian tissue, uterine tissue, colon tissue, prostate tissue, lung tissue, urogenital tissue, stem cells, including cancer stem cell, and body fluids, such as sputum, urine, blood and sweat. In another preferred embodiment, the tissue is selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and tumours. In even another embodiment the sample is selected from the group consisting of breast tissue, ovarian tissue, uterine tissue, colon tissue, prostate tissue and lung tissue. In yet another embodiment the sample is selected from the group consisting of stem cells and cancer stem cells. In an even other embodiment the sample is selected from the group consisting of body fluids, sputum, urine, blood and sweat. In an even further embodiment the sample is selected from the group consisting of ovarian tissue, uterine tissue, colon tissue, and urogenital tissue

In an especially preferred embodiment the sample is skin tissue. In another especially preferred embodiment the sample is epidermal tissue. In another especially preferred embodiment the sample is dermal tissue. In another especially preferred embodiment the sample is blood tissue. In another especially preferred embodiment the sample is lung tissue. In another especially preferred embodiment the sample is skin tissue. In another especially preferred embodiment the sample is prostate tissue. In another especially preferred embodiment the sample is ovarian tissue.

In the analysis in respect of an agent or compound of the present invention, evaluation of the transcriptional and/or translational products is in one embodiment performed in the live animal. In another embodiment, evaluation of the transcriptional and/or translational products is performed without removing the tissue from the live animal. In another embodiment, however, evaluation of the transcriptional and/or translational products is performed on a sample removed from the animal. The sample may be derived from any of the tissues as defined elsewhere herein. For example, the sample is selected from the group consisting of skin tissue, including epidermal and dermal tissue, breast tissue, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and/or sweat.

The methods of the present invention may comprise one or more evaluation steps. In one embodiment, the methods of the present invention further comprise at least one additional evaluation step. Evaluation may take place one or more times, for example the evaluation steps are separated by at least 1, 2, 3, 4, 5, 10, 20, 30, 60, 180, 365, or 700 days.

Vectors

The sensor system and reporter system according to the present invention may be comprised in a one or more recombinant DNA vectors. The vector may be any vector including any commercially available vectors and other vectors known to those of skill within the art. Consequently, the vector is a retroviral vector, a shuttle vector or a mammalian expression vector. In one embodiment, the vector is a Transposon-based vector, such as a vector based on the Sleeping Beauty DNA transposon. In another embodiment, the vector is a Recombinase-based vector, such as specifically, a FLP-FRT recombinase vector.

Administration

The physical or chemical agent and/or compound which may be evaluated by a method or use of the present invention is administered by any appropriate administration method known to those of skill within the art. In one embodiment, administration comprises oral, including buccal and sublingual, rectal, nasal, topical, pulmonary, vaginal, or parenteral, including intramuscular, intraarterial, intrathecal, subcutaneous and intravenous administration or administration by inhalation or insufflation. In a preferred embodiment, the agent is administered by topical administration. In another embodiment, said administration pulmonary administration.

In some cases, the methods of the present invention comprises a repeating the of administration of the agent and/or compound to the tissue. Thus, the agent and/or compound may be administered multiple rounds of administration, such as at least two, for example at least three, for example at least 4, such as at least 5, for example at least 6, such as at least 7, such as at least 8, for example at least 9 such as at least 10, such as at least 20, for example at least 30, for example at least 40, such as at least 50, for example at least 60, such as at least 70, such as at least 80, for example at least 90 such as at least 100 rounds of administration.

The multiple administration rounds is separated by at least 1 hour, such as at least 2 hours, for example at least 3 hours, such as at least 4 hours, for example at least 5 hours, such as at least 6 hours, for example at least 7 hours, such as at least 8 hours, for example at least 9 hours, such as at least 10 hours, for example at least 11 hours, such as at least 12 hours, for example at least 13 hours, such as at least 14 hours, for example at least 16 hours, such as at least 18 hours, for example at least 20 hours, such as at least 22 hours, for example at least 24 hours. In another embodiment, the administration rounds is separated by at least 1 day, such as at least 2 days, for example at least 3 days, such as at least 4 days, for example at least 5 days, such as at least 6 days, for example at least 7 days, such as at least 8 days, for example at least 9 days, such as at least 10 days, for example at least 12 days, such as at least 14 days, for example at least 16 days, such as at least 18 days, for example at least 20 days, such as at least 30 days, for example at least 40 days, such as at least 50 days, for example at least 100 days.

Animals

In one aspect, the present invention relates to a transgenic animal comprising

i. at least one nucleic acid sequence encoding a nuclear receptor or part thereof and a DNA binding domain or part thereof, and

ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or

iii. the transcriptional or translational products of any of said nucleic acid sequences.

In one embodiment, the transgenic animal is selected from the group consisting of pig, minipig, micropig, mouse, rat, non-human primate and rodent. The transgenic animal of the present invention is preferably a pig.

The present invention also relates to a method of evaluating the effect of an agent in a tissue of an animal. Thus, in one aspect the present invention relates to a method for evaluating the effect of an agent on the activity of a nuclear receptor in a tissue of a non-human animal, said method comprising

a. providing a non-human transgenic animal comprising i. at least one nucleic acid sequence encoding a nuclear receptor or part thereof and a DNA binding domain or part thereof, and ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or iii. the transcriptional or translational products of any of said nucleic acid sequences.

b. administering an agent to said transgenic animal, and

c. detecting the expression of said nucleic acid sequence encoding a reporter nucleic acid transcript and/or reporter polypeptide or part thereof in said animal,

wherein the expression upon administration of said agent is indicative of the effect of said agent on the activity of a nuclear receptor in said tissue.

In another aspect, the present invention relates to a method for testing a compound for the ability to alter an effect of an agent on the activity of a nuclear receptor in a tissue of a non-human animal comprising

a. providing a non-human transgenic animal comprising i. at least one nucleic acid sequence encoding a nuclear receptor or part thereof and a DNA binding domain or part thereof, and ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or iii. the transcriptional or translational products of any of said nucleic acid sequences.

b. administering said compound to said transgenic animal,

c. administering said agent to said transgenic animal, and

d. detecting the expression of said nucleic acid sequence encoding a reporter nucleic acid transcript and/or reporter polypeptide or part thereof in said animal,

wherein the expression upon administration of said agent is indicative of the effect of said agent on the activity of a nuclear receptor in said tissue.

In the methods of the present invention, the expression of the nucleic acid sequence encoding a reporter nucleic acid transcript and/or reporter polypeptide or part thereof is detected in the presence and absence of said agent and/or compound. In preferred embodiments of the methods of the present invention, in the presence of said agent compared with the absence of said agent

a. an increase in the expression of said reporter transcript or polypeptide is indicative of a stimulatory effect of said agent on the activity of said nuclear receptor,

b. a decrease in the expression of said reporter transcript or polypeptide is indicative of an inhibitory effect of said agent on the activity of said nuclear receptor, and

c. an unchanged expression of said reporter transcript or polypeptide is indicative of said agent having no or little effect on the activity of said nuclear receptor.

Similarly, in other preferred embodiments of the methods of the present invention, in the presence of said compound compared with the absence of said compound

a. an increase in the effect of said agent on the expression of said reporter transcript or polypeptide is indicative of a stimulatory effect of said compound on the effect of said agent on the activity of said nuclear receptor,

b. a decrease in the effect of said agent on the expression of said reporter transcript or polypeptide is indicative of an inhibitory effect of said compound on the effect of said agent on the activity of said nuclear receptor, and

c. a little or unchanged effect of said agent on the expression of said reporter transcript or polypeptide is indicative of said compound having no or little effect on the effect of said agent on the activity of said nuclear receptor.

In one embodiment, said animal is a human, non-human primates, pig, minipig, micropig, mouse, rat and rodent. In a specific embodiment, said animal is a human being. The effect of an agent in a tissue of an animal including a human being according to the present invention, is evaluated by providing a transgenic animal, comprising at least one nucleic acid sequence, wherein said at least one nucleic acid sequence encodes a reporter polypeptide or part thereof, and/or an additional nucleic acid sequence encodes a fusion polypeptide, comprising a nuclear receptor or part thereof coupled to a DNA binding domain, or the transcriptional or translational products of said additional nucleic acid sequence. The agent is administered to the transgenic animal, and the transcriptional and/or translational expression product of the nucleic acid sequence encoding the reporter polypeptide is evaluated. An alteration of the expression product prior to and after administration of the agent is indicative of an effect on said tissue.

The transgenic animal according to the present invention is a non-human animal, which comprise a foreign gene in its genome. The foreign gene may be comprised in germ line tissue and thus, be transmitted to offspring. Exogenous genes can be transferred to the genome of the animal by techniques known to those of skill within the art. In one embodiment, the transgenic animal of the present invention is selected from the group consisting of non-human primates, pig, minipig, micropig, mouse, rat and rodent. In a preferred embodiment, the transgenic animal is pig (sus scrofus). In another preferred embodiment, the transgenic animal is mouse (mus musculus).

Transgenic animals can be obtained by a number of methods, which are known to those of skill within the art. Examples of such techniques include microcapillary injection into single cell embryos, recombinant techniques using Cre/lox or Flp/FRT systems, and transfection using liposomes or electroporation. The modified genetic material may also be provided by transposition (e.g. by use of Sleeping Beauty transposition). Moreover, viral transduction (e.g. retroviral or lentiviral based vectors) are suitable for the generation of a transgenic animal. In a preferred embodiment, blastocysts for transfer is done by somatic cell nuclear transfer (SCNT), as described elsewhere herein.

In one aspect, the present invention relates to a transgenic animal comprising at least one nucleic acid sequence, wherein i. said at least one nucleic acid sequence encodes a reporter polypeptide or part thereof, and/or ii. an additional nucleic acid sequence encodes a fusion polypeptide, comprising a nuclear receptor or part thereof coupled to a DNA binding domain, or the transcriptional or translational products of said additional nucleic acid sequence. That transgenic animal is suitable for evaluating an agent for its effect on a tissue and/or for testing a compound for the ability to alter the effects of an agent in a tissue of that animal. In one embodiment, the animal is selected from the group consisting of pig, mouse, rat, rodent, dog, monkey, guinea pig, minipig and/or micropig. In a preferred embodiment, the transgenic animal is a pig. In another embodiment, the animal is a mouse.

The reporter polypeptide of the transgenic animal may be selected from any reporter polypeptide described herein. In one example the reporter polypeptide of the transgenic animal is selected from the group consisting of β-galactosidase, HcRed, DsRed, DsRed monomer, ZsGreen, AmCyan, ZsYellow, fire fly luciferase, renilla luciferase, SEAP, EGFP, EBFP, EYFP, d2EGFP and GFPuv, cyan, green yellow, red, and far red Reef Coral Fluorescent Protein and/or fragments, modifications and/or functional variants thereof. In a preferred embodiment, reporter polypeptide is β-galactosidase or a fragment or functional variant thereof.

Also, the nuclear receptor of the transgenic animal may be selected from any of those defined elsewhere herein. In one embodiment, the nuclear receptor is selected from the group consisting of vitamin D receptor, Liver X receptors, promiscuous pregnane X receptor and/or PPARs, and/or a fragment thereof, in particular ligand-binding domains.

The DNA binding domain of the transgenic animal is preferably selected from the group consisting of GAL4 DNA binding domain and LexA DNA binding domain, however, any DNA binding domain may be selected for this purpose.

In a specific embodiment, the present invention relates to a transgenic pig, comprising at least one nucleic acid sequence, wherein a. said at least one nucleic acid sequence encodes β-galactosidase or part thereof, and/or b. an additional nucleic acid sequence encodes a fusion polypeptide, comprising PPARδ or part thereof coupled to yeast GAL4 DNA binding domain, or the transcriptional or translational products of said additional nucleic acid sequence.

A transgenic animal, such as a transgenic pig, of the present invention may be used for determining in vivo the activation of nuclear receptors due to the production of endogenous agonists. In one example, the agonists are generated during normal development of the skin, however, in another embodiment, the endogenous agonists are generated during the development of a disease, such is psoriasis, different cancer types and/or other hyperproliferative diseases. In a preferred embodiment, the disease is psoriasis.

A transgenic animal, such as a transgenic pig, of the present invention is suitable for determining penetration of an agent in situ in a tissue and/or the activation of nuclear receptors by an agent, such as an agent as defined elsewhere herein.

In another aspect, the present invention relates to a cell line derived from any transgenic animal as defined herein. The term derived is meant to indicate that the cell line is based on a cell from a transgenic animal of the present invention. the cell may be further modified after isolation from the transgenic animal to develop an modified transgenic cell line.

In one aspect, the present invention relates to a transgenic non-human oocyte, sperm cell, blastocyst, embryo, fetus, donor cell, or cell nucleus derived from the transgenic non-human animal of the present invention, and/or a transgenic non-human oocyte, sperm cell, blastocyst, embryo, fetus, donor cell, or cell nucleus, wherein the transgenic genome comprises

i. at least one nucleic acid sequence encoding a nuclear receptor or part thereof and a DNA binding domain or part thereof, and

ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or

iii. the transcriptional or translational products of any of said nucleic acid sequences,

The present invention also in one aspect relates to the production of a transgenic animal of the present invention, in particular a transgenic pig, as well as methods of producing an oocyte, sperm cell, blastocyst, embryo, fetus, donor cell, or cell nucleus of the present invention. Thus, in one aspect, the present invention relates to a method of producing a transgenic non-human animal, oocyte, sperm cell, blastocyst, embryo, fetus, donor cell, or cell nucleus of the invention comprising the steps of

i. providing a donor cell,

ii. genetically modifying the donor cell of i) by inserting a. at least one nucleic acid sequence encoding a nuclear receptor or part thereof and a DNA binding domain or part thereof, and b. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or c. the transcriptional or translational products of any of said nucleic acid sequences,

iii. transferring the modified genome of the donor cell obtained in ii) into a host cell,

iv. obtaining a reconstructed embryo forming an embryo

v. culturing said embryo; and

vi. transferring said cultured embryo to a host mammal such that the embryo develops into a genetically modified fetus,

wherein said genetically modified embryo is produced by nuclear transfer comprises steps i) to v),

wherein said genetically modified blastocyst is produced by nuclear transfer comprises steps i) to vi),

wherein said genetically modified fetus is produced by nuclear transfer comprises steps i) to vi).

For the production of a transgenic animal such as a transgenic pig of the present invention, the donor (somatic cell or nucleus of somatic cell) and recipient (cytoplast) involved in the cell nuclear transfer method according to the present invention is a non-human mammal. Likewise, the animal in which reconstructed embryos may be implanted in according to the present invention is a non-human mammal, preferably a.

The mammal may be an ungulate selected from the group consisting of domestic or wild representatives of bovidae, ovids, cervids, suids, equids and camelids. In a particular embodiment the mammal is a cow or bull, bison, buffalo, sheep, big-horn sheep, horse, pony, donkey, mule, deer, elk, caribou, goat, water buffalo, camel, llama, alpaca or pig. In a special embodiment of the present invention the mammal is a pig. In one embodiment the pig is a wild pig, In another embodiment the pig is the domestic pig Sus scrofa, or S. domesticus. In yet another embodiment the invention relates to mini pig, but also to inbred pigs. In a specific embodiment the pig may be selected from the group consisting of Landrace, Yorkshire, Hampshire, Duroc, Chinese Meishan, Berkshire and Piêtrain. In yet another embodiment the present invention relates to the group consisting of Landrace, Yorkshire, Hampshire and Duroc. However the present invention also relates to the group consisting of Landrace, Duroc and Chinese Meishan. Similarly, the group consisting of Berkshire, Pietrain, Landrace and Chinese Meishan can be objects of the Present invention. But also the group consisting of Landrace and Chinese Meishan are objects of the present invention. In a particular embodiment the pig is a Landrace pig, or a Yorkshire pig. In a particular embodiment the invention relates to pigs of the breed Hampshire, but also Duroc. In yet another preferred embodiment the pig is of the breed Chinese Meishan. However, also Berkshire is covered by the invention, and in a special embodiment Piêtrain is covered by the present invention. Another embodiment of the present invention relates to mini pigs selected from the group consisting of Goettingen, Yucatan, Bama Xiang Zhu, Wuzhishan , Xi Shuang Banna. In other embodiments the invention relates to the group consisting of Goettingen, Yucatan. Alternatively, the invention relates to the group consisting of Bama Xiang Zhu, Wuzhishan, Xi Shuang Banna. In particular the invention relates to Goettingen. But also Yucatan is relevant for the invention. Similarly, Bama Xiang Zhu is covered by the invention, also Wuzhishan, and in particular Xi Shuang Banna. The donor mammals according to the present invention may be female, or male. The age of the mammal can be any age such as an adult, or for example a fetus.

Transgenic Pig and Cloning of the Pig

The transgenic pig according to the present invention comprises at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence.

In one embodiment, the transgenic pig comprising at least one nucleic acid sequence, wherein i) said at least one nucleic acid sequence encodes a reporter polypeptide or part thereof, and/or ii) an additional nucleic acid sequence encodes a fusion polypeptide, comprising a nuclear receptor or part thereof coupled to a DNA binding domain, or the transcriptional or translational products of said additional nucleic acid sequence, is obtained by crossing a transgenic pig comprising at least one nucleic acid sequence, wherein said at least one nucleic acid sequence encodes a reporter polypeptide or part thereof with a transgenic pig comprising at least one nucleic acid sequence, wherein said at least one nucleic acid sequence encodes a fusion polypeptide, comprising a nuclear receptor or part thereof coupled to a DNA binding domain, or the transcriptional or translational products of said additional nucleic acid sequence.

Thus, the present invention also relates to a transgenic pig comprising at least one nucleic acid sequence, wherein said at least one nucleic acid sequence encodes a reporter polypeptide or part thereof.

In another embodiment, the present invention relates to a transgenic pig comprising at least one nucleic acid sequence, wherein said at least one nucleic acid sequence encodes a fusion polypeptide, comprising a nuclear receptor or part thereof coupled to a DNA binding domain, or the transcriptional or translational products of said additional nucleic acid sequence.

By use of state of the art pig cloning and gene transfer technology it is possible to integrate the reporter systems and/or sensor systems, such as i. at least one nucleic acid sequence encoding a nuclear receptor or part thereof and a DNA binding domain or part thereof, and ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or into the nuclei of pig fibroblasts, which subsequently are used for transfer into egg cytoplasts.

In a preferred embodiment, the transgenic pigs according to the present invention are produced via cloning by somatic cell nuclear transfer from genetically engineered fibroblasts to egg cytoplasts. Thereby, the genetic reporter system and/or sensor system described elsewhere herein is integrated in the genome to obtain transgenic reporter and/or sensor pigs, respectively. Specifically, somatic cell nuclear transfer is conducted by handmade cloning (HMC), such as described by Gabor Vajta (trends in biotechnology, 2007).

Pig strains comprising the nuclear receptor sensor system according to the present invention can be used to determine in vivo the activation of nuclear receptors due to the production of endogenous agonists. In one embodiment, the endogenous agonists are generated during normal development of the skin. In another embodiment, the endogenous agonists are generated during the development of a disease state. In a specific embodiment, said disease is psoriasis, different cancer types and/or other hyperproliferative diseases. In one embodiment, said disease is any skin disease. In another embodiment, said disease is any cancer disease. In a preferred embodiment, said disease is psoriasis. In another preferred embodiment, said disease is skin cancer.

In another aspect of the present invention, the pig strains comprising the nuclear receptor sensor system according to the present invention can be used to study penetration in situ of a tissue and the activation nuclear receptors by agents, including xenobiotics.

In a specific embodiment, transgenic pig strains comprise fusions of the GAL4 DNA binding domain or LexA DNA binding domain and the ligand binding domains of the PXR, retinoic acid receptor, the vitamin D receptor or any of the PPARs integrated into the genome and expressed in a specific tissue. In a preferred embodiment, this tissue is skin, epidermis, dermis, hypoderm or the basal cells of the epidermis. In another specific embodiment, the Keratin 14 enhancer/promoter is integrated upstream of the gene encoding the fusion polypeptide to drive skin specific expression.

In an additional embodiment, the transgenic pig comprise at least one nucleic acid sequence, wherein said at least one nucleic acid sequence encodes a fusion polypeptide, comprising the ligand binding domains of the Pregnane X receptor or part thereof coupled to the GAL4 DNA binding domain or LexA DNA binding domain, or the transcriptional or translational products of said nucleic acid sequence. In another embodiment, the transgenic pig comprise at least one nucleic acid sequence, wherein said at least one nucleic acid sequence encodes a fusion polypeptide, comprising the ligand binding domains of the retinoic acid receptor or part thereof coupled to the GAL4 DNA binding domain or LexA DNA binding domain, or the transcriptional or translational products of said nucleic acid sequence. In yet another embodiment, the transgenic pig comprise at least one nucleic acid sequence, wherein said at least one nucleic acid sequence encodes a fusion polypeptide, comprising the ligand binding domains of the the vitamin D receptor or part thereof coupled to the GAL4 DNA binding domain or LexA DNA binding domain, or the transcriptional or translational products of said nucleic acid sequence. In an additional embodiment, the transgenic pig comprise at least one nucleic acid sequence, wherein said at least one nucleic acid sequence encodes a fusion polypeptide, comprising the ligand binding domains of any of the PPARs, including PPARδ or part thereof coupled to the GAL4 DNA binding domain or LexA DNA binding domain, or the transcriptional or translational products of said nucleic acid sequence.

Somatic Cell Nuclear Transfer

In cloning, the transfer of the nucleus of a somatic (body) cell or somatic cell into an egg cell (oocyte) which has had its own nucleus removed (denucleated or enucleated) is called somatic cell nuclear transfer (SCNT). The new individual will develop from this reconstructed embryo and be genetically identical to the donor of the somatic cell. In the present invention the method of somatic cell nuclear transfer is a method of cell nuclear transfer comprising the steps of a) providing at least one oocyte having at least a part of a modified zona pellucida, b) separating the oocyte into at least two parts obtaining at least one cytoplast, c) providing at least one a donor cell or cell nucleus having desired genetic properties, d) fusing at least one cytoplast with the donor cell or membrane surrounded cell nucleus. However, the present invention also relates to a method of cell nuclear transfer comprising the steps of a) providing at least one oocyte, b) separating the oocyte into at least three parts obtaining at least two cytoplasts, c) providing at least one a donor cell or cell nucleus having desired genetic properties, d) fusing at least one cytoplast with the donor cell or membrane surrounded cell nucleus. The parameters for the listed steps can be varied in order to obtain the most efficient nuclear transfer for a given animal species. The various parameters are described in detail below.

Oocyte

The term ‘oocyte’ according to the present invention means an immature female reproductive cell, one that has not completed the maturing process to form an ovum (gamete). In the present invention an enucleated oocyte is the recipient cell in the nuclear transfer process.

The oocytes according to the present invention are isolated from oviducts and/or ovaries of a mammal. Normally, oocytes are retrieved from deceased animals, although they may be isolated also from either oviducts and/or ovaries of live animals. In one embodiment the oocytes are isolated by oviductal recovery procedures or transvaginal recovery methods. In a preferred embodiment the oocytes are isolated by aspiration. Oocytes are typically matured in a variety of media known to a person skilled in the art prior to enucleation. The oocytes can also be isolated from the ovaries of a recently sacrificed animal or when the ovary has been frozen and/or thawed. Preferably, the oocytes are freshly isolated from the oviducts.

Oocytes or cytoplasts may also be cryopreserved before use. While it will be appreciated by those skilled in the art that freshly isolated and matured oocytes are preferred, it will also be appreciated that it is possible to cryopreserve the oocytes after harvesting or after maturation. If cryopreserved oocytes are utilised then these must be initially thawed before placing the oocytes in maturation medium. Methods of thawing cryopreserved materials such that they are active after the thawing process are well-known to those of ordinary skill in the art. However, in general, cryopreservation of oocytes and cytoplasts is a very demanding procedure, and it is especially difficult in pigs, because of the abovementioned general fragility of pig oocytes and cytoplasts, and because of the high lipid content that makes them very sensitive to chilling injury (i.e. injury that occurs between +15 and +5 degrees C. during the cooling and warming procedure).

In another embodiment, mature (metaphase II) oocytes that have been matured in vivo, may be harvested and used in the nuclear transfer methods disclosed herein. Essentially, mature metaphase II oocytes are collected surgically from either nonsuperovulated or superovulated mammals 35 to 48 hours past the onset of estrus or past the injection of human chorionic gonadotropin (hCG) or similar hormone.

Where oocytes have been cultured in vitro, cumulus cells that are surrounding the oocytes in vivo may have accumulated may be removed to provide oocytes that are at a more suitable stage of maturation for enucleation. Cumulus cells may be removed by pipetting or vortexing, for example, in the presence of in the range of 0.1 to 5% hyaluronidase, such as in the range of 0.2 to 5% hyaluronidase , for example in the range of 0.5 to 5% hyaluronidase, such as in the range of 0.2 to 3% hyaluronidase, for example in the range of 0.5 to 3% hyaluronidase, such as in the range of 0.5 to 2% hyaluronidase, for example in the range of 0.5 to 1% hyaluronidase, such as 0.5% hyaluronidase.

The first step in the preferred methods involves the isolation of a recipient oocyte from a suitable animal. In this regard, the oocyte may be obtained from any animal source and at any stage of maturation.

The stage of maturation of the oocyte at enucleation and nuclear transfer has been reported to be of significance for the success of nuclear transfer methods. Immature (prophase I) oocytes from mammalian ovaries are often harvested by aspiration. In order to employ techniques such as genetic engineering, nuclear transfer and cloning, such harvested oocytes are preferably matured in vitro before the oocyte cells may be used as recipient cells for nuclear transfer.

Preferably, successful mammalian embryo cloning uses the metaphase II stage oocyte as the recipient oocyte because it is believed that at this stage of maturation the oocyte can be or is sufficiently activated to treat the introduced nucleus as if it were a fertilising sperm. However, the present invention relates to any maturation stage of the oocyte which is suitable for carrying out somatic cell nuclear transfer, embryos, blastocysts, and/or animals obtainable by the method of somatic cell nuclear transfer of the present invention. The in vitro maturation of oocytes usually takes place in a maturation medium until the oocyte has reached the metaphase II stage or has extruded the first polar body. The time it takes for an immature oocyte to reach maturation is called the maturation period. In a preferred embodiment of the present invention the oocyte is from sow or gilt, preferably from a sow.

Embryo

According to the present invention a reconstructed embryo (i.e. single cell embryo) contains the genetic material of the donor cell. Subsequently, the reconstructed embryo divides progressively into a multi-cell embryo after the onset of mitosis. In vitro the onset of mitosis is typically induced by activation as described herein.

In the present invention the term ‘embryo’ also refers to reconstructed embryos which are embryos formed after the process of nuclear transfer after the onset of mitosis by activation. Reconstructed embryos are cultured in vitro.

When the embryo contains about 12-16 cells, it is called a “morula”. Subsequently, the embryo divides further and many cells are formed, and a fluid-filled cystic cavity within its center, blastocoele cavity. At this stage, the embryo is called a “blastocyst”. The developmental stage of the “fertilized” oocyte at the time it is ready to implant; formed from the morula and consists of an inner cell mass, an internal cavity, and an outer layer of cells called trophectodermal cells.

The blastocyst according to the present invention may be implanted into the uterus of a host mammal and continues to grow into a fetus and then an animal.

In the methods provided herein for producing genetically modified or transgenic non-human mammal, for cloning a non-human mammal, for culturing a reconstructed embryo, and /or for cryopreservation of a pig embryo, the embryo may be cultured in vitro. The embryo may for example be cultured in sequential culture. It will be appreciated that the embryo may be a normal embryo, or a reconstructed embryo as defined elsewhere herein.

Cytoplast

An oocyte or a part of an oocyte from which the nucleus has been removed.

Donor Cell

By the term ‘donor cell’ of the present invention is meant somatic cell and/or cells derived from the germ line.

By the term ‘somatic cell’ of the present invention is meant any (body) cell from an animal at any stage of development. For example somatic cells may originate from fetal or adult tissue. Especially preferred somatic cells are those of foetal origin. However, cells from a germ line may also be used. According to the present invention a donor cell is a somatic cell. In another embodiment of the present invention the donor cell is a cell derived from a germ cell line.

In a preferred embodiment of the present invention the donor cell harbours desired genetic properties. However, the donor cell may harbour desired genetic properties which have been gained by genetic manipulation as described elsewhere herein. Somatic cells are selected from the group consisting of epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells.

These may be obtained from different organs, e.g., skin, lung, pancreas, liver, stomach, intestine, heart, reproductive organs, bladder, kidney, urethra and other urinary organs.

The animals from which the somatic cells may be derived are described elsewhere herein. A preferred embodiment of the invention is the use of somatic cells originating from the same species as the recipient oocyte (cytoplast).

Preferably, the somatic cells are fibroblast cells as the can be obtained from both developing fetuses and adult animals in large quantities. Fibroblasts may furthermore be easily propagated in vitro. Most preferably, the somatic cells are in vitro cultured fibroblasts of foetal origin.

In a preferred embodiment the somatic cells are genetically modified. In yet a further preferred embodiment of the present invention the somatic cells are pig cells, and preferably of foetal origin, or for example from adults.

Transgenic Cell Line

The present invention also relates to cell line derived from any of the transgenic animals described herein. Thus, a cell line of the present invention comprise at least one nucleic acid sequence, wherein i. said at least one nucleic acid sequence encodes a reporter polypeptide or part thereof, and/or ii. an additional nucleic acid sequence encodes a fusion polypeptide, comprising a nuclear receptor or part thereof coupled to a DNA binding domain, or the transcriptional or translational products of said additional nucleic acid sequence. Examples of particular reporter polypeptides, nuclear receptors, fusion polypeptides, promoters etc are provided elsewhere herein.

Tissue

The present invention provides a transgenic animal, oocyte, sperm cell, blastocyst, embryo, fetus, donor cell, or cell nucleus, as well as methods and uses for evaluating the effect of a physical and/or chemical agent on the activity of a nuclear receptor a tissue in a tissue. The present invention can be practised on a number of tissues. In one embodiment, the tissue is selected from the group consisting of skin, muscle, lever, lung, tumour and cornea. In another embodiment, the tissue is selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and tumours. In a preferred embodiment of the method of the present invention relates to skin tissue, i.e. said tissue is skin. In a specifically preferred embodiment of the method of the present invention relates to epidermal tissue, i.e. said tissue is epidermis. In another specifically preferred embodiment of the method of the present invention relates to dermal tissue, i.e. said tissue is dermis.

The nuclear receptor sensor system of the present invention can be expressed in a number of tissues. A preferred tissue for detection of activation of nuclear receptors according to the present invention is skin. Skin is comprised of two main layers, the epidermis and the dermis, which is embedded on top of the hypoderm (subcutaneous tissue), comprising Fibroblasts, Adipose Cells, and Macrophages. The upper skin layer, the epidermis, consists of stratified layers of epithelium, wherein cells are formed through mitosis in the deepest layer and migrates to the surface, replacing cells which are continuously sloughed off. Migrating through the epidermal layers, the cells change shape and composition as they differentiate and become filled with keratin, in a process called keratinisation. The outermost layer of epidermis consists of approximately 25 layers of dead cells. The epidermis may be divided into five distinct layers: stratum corneum, stratum lucidum, stratum granulosum, stratum spinosum, and stratum germinativum (or stratum basale, the basal layer). The stratum corneum consists of dead denucleated keratinocytes in which cross-linked structural proteins provides mechanical protection. Keratinocytes in the underlying layer, the stratum granulosum synthesize large quantities of lipids that are required to form a nearly water impermeable barrier, the permeability barrier. The lipids that make up the permeability barrier are mainly cholesterol, fatty acids and ceramides. The lipids are assembled in numerous lipid containing lamellar bodies in the fully differentiated keratinocytes in the stratum granulosurn and then released by exocytosis. After release the lipids are processed and reorganized to form the continuous matrix of lamellar unit structures that make up the functional permeability barrier (Madison, K. C. (2003). Barrier function of the skin: “la raison d'ete” of the epidermis. J Invest Dermatol 121: 231-241). The permeability barrier protects the body against water loss, but at the same time it reduces uptake of biological active molecules administered by topical application. Various liposome-based formulations have been used for delivery of drugs through the skin, and novel formulations based on so-called flexible liposomes have been shown to penetrate deep and efficiently into skin. Application of vesicles to rat skin in vivo: a confocal laser scanning microscopy study. J Control Release 56: 189-96; van Kuijk-Meuwissen, M. E., Junginger, H. E. and Bouwstra, J. A. (1998). Interaction between liposomes and human skin in vitro, a confocal laser scanning microscopy study. Biochim Biophys Acta 1371: 31-9.). Recently, nanoparticles have also been considered as vehicles securing efficient skin penetration.

Dermis is the layer of skin beneath the epidermis. The dermis consists of connective tissues and shields the body from different types of stress and strain. The dermis also contains nerve fibres for sense of touch and heat. It further contains hair follicles, sweat glands, sebaceous glands, apocrine glands and blood vessels. The blood vessels nourish and provide waste removal to the dermal cells as well as the Stratum germinativum of the epidermis.

Information on the activation of nuclear receptors in the different layers of the skin can be obtained by targeting the nuclear receptor sensor system of the present invention into skin of a transgenic animal, such as transgenic cloned pigs, thereby establishing an in vivo model for human skin. The organization of pig skin resembles human skin, thus making pig skin a good model for human skin.

Thus, In a preferred embodiment the nuclear receptor sensor system according to the present invention is incorporated into skin tissue. In another preferred embodiment, the nuclear receptor sensor system according to the present invention is incorporated into epidermal tissue. In another preferred embodiment, the nuclear receptor sensor system according to the present invention is incorporated into dermal tissue.

Agents and Compounds

The present invention offers a method for evaluating the effect of an agent in a tissue of an animal. The invention also relates to a method for testing a compound for the ability to alter the effects of an agent in a tissue of an animal.

The agent and/or compound according to the present invention comprises any possible physical or chemical agent, compound, mixture, composite, complex, substance, material, matter, particle, element, unit, constituent or formulation. In one embodiment, the agent and/or compound is a pharmaceutical composition, cosmetic, drug, xenobiotic compound, food composition, sugar, lipid, protein, dietary supplement, radiation, or electrical stimuli.

In a preferred embodiment, the agent and/or compound is a xenobiotic compound. The term “xenobiotic compound” as used herein refers to any chemical compound, which is not a natural component of the organism exposed to it. Xenobiotic compounds are also known as foreign or exogenous substances or compounds. Xenobiotic compounds also cover naturally occurring substances which are present in much higher concentrations than are usual.

Examples of xenobiotic compound include without limitation naturally occurring compounds, drugs, antibiotics, environmental agents, pollutants such as dioxins and polychlorinated biphenyls, carcinogens, and insecticides. In a preferred embodiment, the agent and/or compound is a vitamin D analog.

In another preferred embodiment, the agent and/or compound is radiation, including ultraviolet radiation (UV-radiation), infrared radiation, electromagnetic radiation, gamma-radiation (γ-radiation), x-rays, and sunshine.

In a specifically preferred embodiment, the agent and/or compound is UV-radiation.

In another preferred embodiment, the agent and/or compound is a cosmetic, such as a skin lotion, sun lotion or sun block lotion. More specifically, in one embodiment, the agent is UV-radiation, such as UV-C radiation and the compound is a sun lotion or sun block lotion. In this way, the transgenic animal, cells, methods and uses of the present invention can be used to evaluate the ability of a sun lotion/block composition to counteract the effects of UV-radiation on the activity of a nuclear receptor in a tissue, such as a skin tissue.

The agents and compounds of the present invention comprise any shape, size or conformation. In one embodiment, the agent is in the form of fluids, crystals, solutions, crèmes, lotions, gels, microparticles, or nanoparticles.

Specific Applications

The methods, animals and cell lines of the present invention can be used for a number of specific applications.

In one embodiment, the methods, animals and cell lines of the present invention can be used for evaluation of the effect of a physical or chemical agent in a tissue, such as skin tissue, on the activation of a specific nuclear receptor. Such effects can be used for interpretation of the ability of an agent to penetrate the specific tissue. This aspect of the invention is covered by the method for evaluating the effect of an agent in a tissue of an animal comprising a. providing a transgenic animal, comprising at least one nucleic acid sequence, wherein i. said at least one nucleic acid sequence encodes a reporter polypeptide or part thereof, and/or ii. an additional nucleic acid sequence encodes a fusion polypeptide, comprising a nuclear receptor or part thereof coupled to a DNA binding domain, or the transcriptional or translational products of said additional nucleic acid sequence, b. administering said agent to said animal, and c. evaluating the transcriptional and/or translational expression product of the nucleic acid sequence encoding the reporter polypeptide, wherein an alteration of said expression product prior to and after step (b) is indicative of an effect on said tissue.

In another embodiment, the methods, animals and cell lines of the present invention can be used for evaluation of the ability of a compound to counteract or enhance the effect of a physical or chemical agent in a tissue. In one embodiment, such a compound is a sun lotion. This aspect of the invention is covered by the method for testing a compound for the ability to alter the effects of an agent in a tissue of an animal comprising a. administering said compound to a transgenic animal comprising at least one nucleic acid sequence, wherein i. said at least one nucleic acid sequence encodes a reporter polypeptide or part thereof, and/or ii. an additional nucleic acid sequence encodes a fusion polypeptide, comprising a nuclear receptor or part thereof coupled to a DNA binding domain, or the transcriptional or translational products of said additional nucleic acid sequence, b. administering said agent to said transgenic animal, and c. evaluating the transcriptional and/or translational expression product of the nucleic acid sequence encoding the reporter polypeptide, wherein a difference in the amount of said expression product in the presence and absence of said compound is indicative of said compound being able to alter the effect of said agent in said tissue. The compound of this latter aspect may be any physical or chemical agent, as specified elsewhere herein. In a one embodiment, the compound is selected from the group consisting of a pharmaceutical composition, cosmetic, drug, xenobiotic compound, food composition, sugar, lipid, dietary supplement, radiation and/or electrical stimuli. In a specific embodiment, the compound is in the form of solutions, crèmes, lotions, gels, microparticles and/or nanoparticles. In a preferred embodiment, the compound is a sunlotion. In another embodiment, the agent of the latter aspect is radiation, for example the agent is UV-radiation.

Thus, in one aspect, the present invention relates to use of a non-human transgenic animal, a cell line, an oocyte, sperm cell, blastocyst, embryo, fetus, donor cell, and/or cell nucleus of the present invention for evaluating the activity of a nuclear receptor. In a preferred embodiment, the use relates to evaluating the effect of an agent on the activity of a nuclear receptor, for example an agent as defined above. Thus, the animal, a cell line, an oocyte, sperm cell, blastocyst, embryo, fetus, donor cell, and/or cell nucleus of the present invention may be used for evaluating the effect of a physical or chemical agent on the activity of a nuclear receptor, by comparing the expression on a reporter transcript and/or polypeptide in the presence and absence of said agent, as described elsewhere herein. In another embodiment, the use relates the evaluating in vivo the activity of a nuclear receptor due to endogenous agonists, for example due to agonists that are generated during normal development of the skin. In this way, the temporal or spatial activation of a specific nuclear receptor may be evaluated by detecting a temporal-spatial expression of reporter transcript and/or polypeptide. In a specific embodiment, however, the endogenous agonists are generated during the development of a disease, such as psoriasis, different cancer types and/or other hyperproliferative diseases.

### EXAMPLES

To obtain information on the activation of nuclear receptors in the different layers of the skin, genetic reporter systems are designed that can be targeted to the skin of transgenic cloned pigs to establish an alternative in vivo model for human skin. The nuclear receptor sensor systems can be used for three purposes. Firstly, the invention can be used to examine the ability of different types of liposomes or other formulations to transport compounds into the skin. By treatment of the skin with a formulation comprising a nuclear receptor activator, activation of the reporter will reflect the penetration ability of the formulation. Secondly, the sensor-cell system allows examination of the ability of various xenobiotics to penetrate into the epidermis. Finally, the sensor-cell system also allows determination of the activation of nuclear receptors due to the production of endogenous agonists during normal development and skin homeostasis and/or during different disease stages.

### Example 1

The Nuclear Receptor Sensor Systems

The reporter system consists of a cassette containing an enhancer/promoter that drives expression of a reporter gene. The conventional P-galactosidase gene is used, thus allowing simple enzymatic detection of expression. Subsequently, reporters based on the use of green fluorescent protein (or various derivatives of the green fluorescent protein) are used to enable direct fluorescent detection by confocal and multiphoton fluorescent microscopy. The enhancer/promoter is conventional combinations of the yeast UAS(gal) enhancer or the bacterial LexA binding site fused with the thymidin kinase promoter. To activate these reporter systems, fusions between the yeast GAL4 DNA binding domain or the LexA DNA binding domain and the ligand binding domains of the retinoic acid receptor, the vitamin D receptor, the liver X receptors, activate the promiscuous pregnane X receptor and the PPARs will be used. To ensure skin-specific expression, the promoter regions of these constructs will be replaced by the keratin 14 enhancerlpromoter, a promoter known to drive epidermis specific expression. For production of sensor cell lines and transgenic cloned pigs PXR is used—being a likely target for numerous xenobiotics—and also PPARdelta—the main PPAR subtype expressed in human epidermis—the vitamin D receptor—a known pharmaceutical target in the treatment of psoriasis—and the retinoic acid receptor—a validated skin target and regulator of skin homeostasis.

### Example 2

Transgenic Pig as a Model for Testing of Penetration of Pharmaceuticals and Xenobiotics into the Skin

Pig skin is a good model for human skin. The transgenic pigs are based on cloning by somatic cell nuclear transfer from genetically engineered fibroblasts to egg cytoplasts. By this approach the genetic reporter system described of the present invention is integrated to the genome to obtain transgenic reporter pigs. Further, transgenic pig strains will be generated in which the fusions of the GAL4 DNA binding domain or LexA DNA binding domain and the ligand binding domains of the PXR, retinoic acid receptor, the vitamin D receptor and the PPARs are integrated into the genome and expressed in the basal cells of the epidermis by using the K14 enhancer/promoter to drive skin-specific expression. Crossing the transgenic reporter pigs with the transgenic K14-nuclear receptor transgenic pigs will generate the sensor pig strains. These sensor pig strains can be used to determine activation due to the production of endogenous agonists that are generated during normal development of the skin. Furthermore, the sensor pig strains can also be used to study skin penetration in situ and the nuclear receptor activation by xenobiotics. Analysis of skin penetration is subsequently performed as described below.

For example the ability of different formulations to promote the penetration of test compounds into the skin is analyzed as well as the penetration of various xenobiotics as determined by the activation of nuclear receptors. The spatial activation of nuclear receptors in response to exposure to the selected pharmaceuticals in various formulations and xenobiotics can be determined. The read out is the induction of reporter genes expressing enzymes detected by immunohistochemistry or reporters expressing fluorescent proteins detected by confocal fluorescence and/or multi-photon excitation fluorescence microscopy. In addition, the penetration of various liposome and nanoparticle formulations in the skin can be examined directly by confocal fluorescence and/or multi-photon excitation fluorescence microscopy of extrinsic fluorescent probes, whereby a direct correlation between the spatial distribution of the formulation and skin structure can be obtained. Confocal fluorescence and/or multi-photon excitation fluorescence microscopy has successfully been used to ascertain dynamical and structural information about the skin. For instance, the sectioning capabilities of these two techniques are very valuable to disentangle the complex 3D structure of the skin tissue in a non-invasive way, e.g. by using naturally occurring or extrinsic fluorescent probes. Both in vivo and ex vivo imaging of dermal and subcutaneous structures of animal and human skin are available for this purpose.

### Example 3

Sensor-Receptor System Transient In Vitro

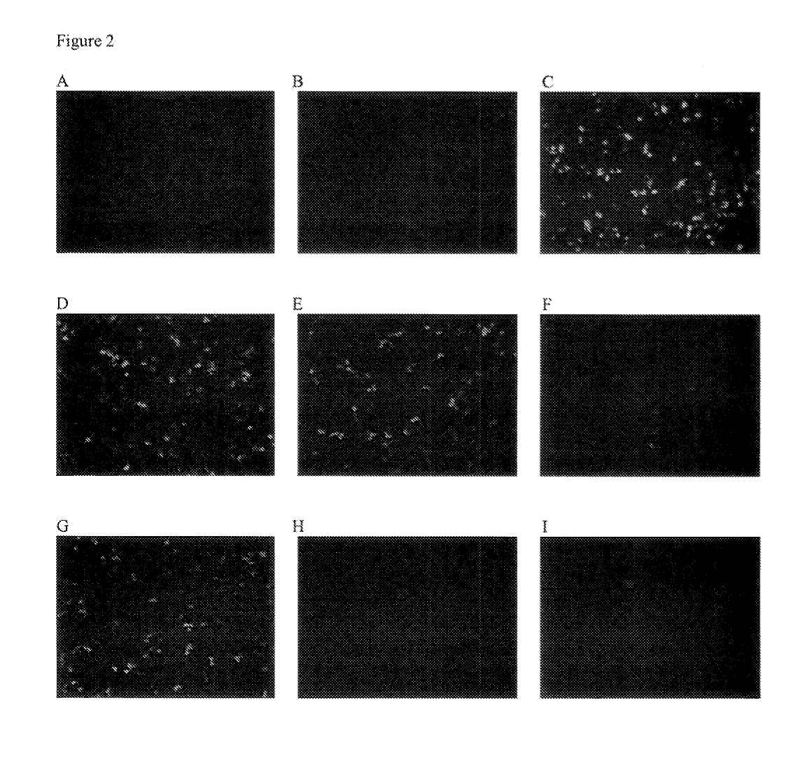

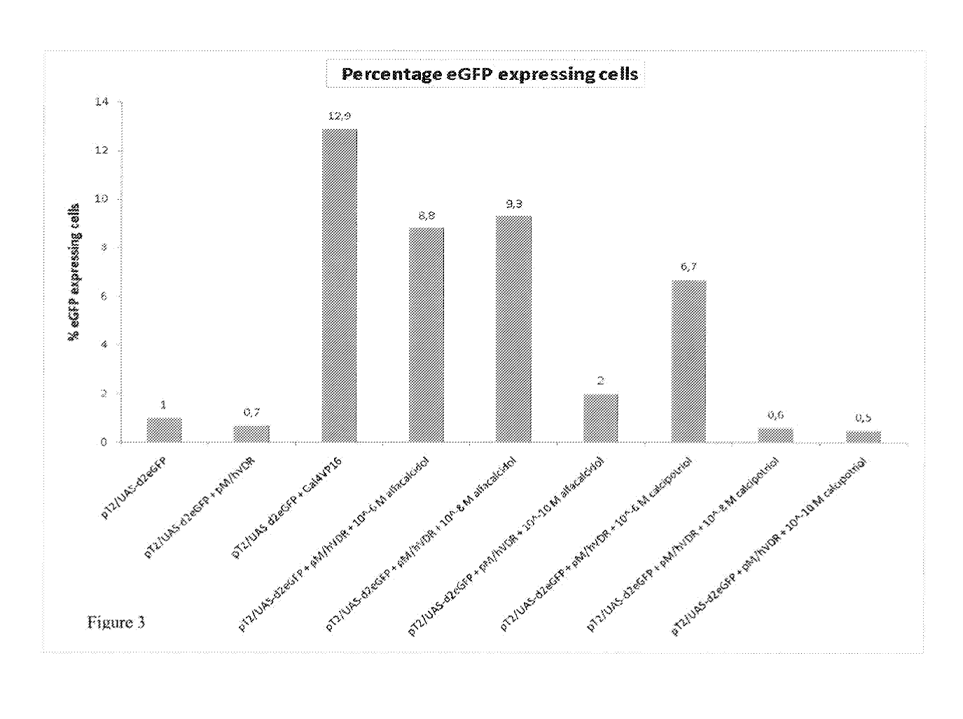

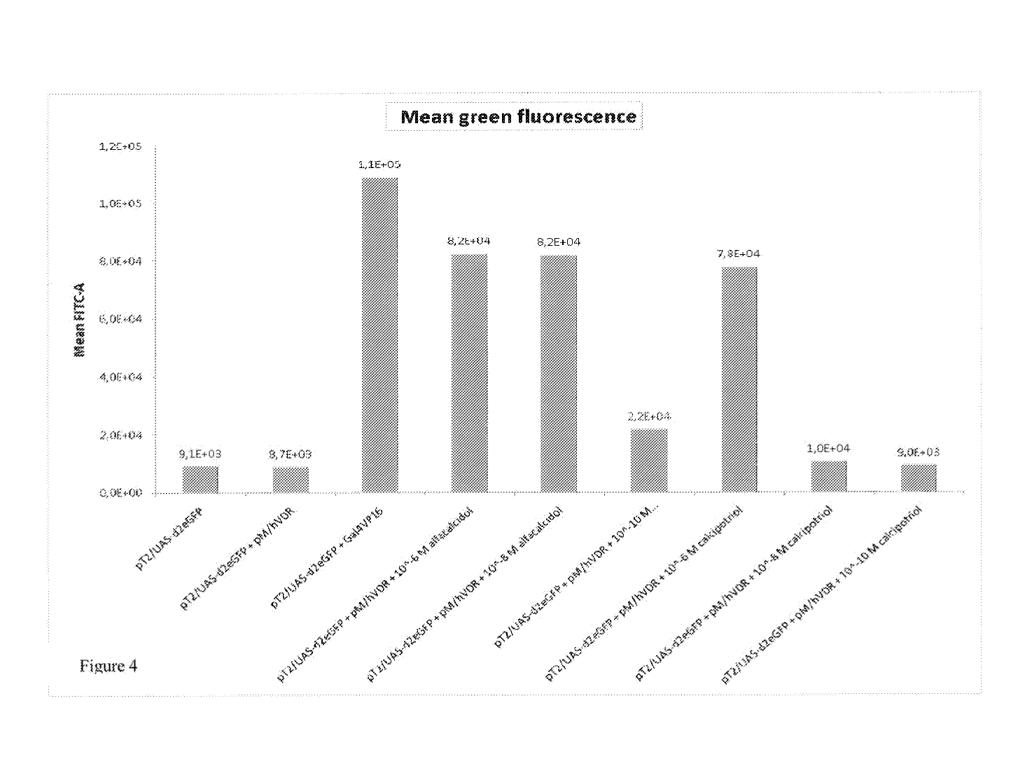

HEK cells were transfected with 1.0 μg of the vectors pT2/UAS-d2eGFP og 1.0 μg pM/hVDR or Gal4VP16. Vitamine D analog was supplied to the cells 12 hours prior to transfection, absent from transfection and the suppied again 3 hours post-transfektion. The cells were analysed by fluorescence microscopy and flow cytometry 24 hours post-transfektion, see FIGS. 2-4.

Description of Vectors:

pT2/UAS-d2eGFP comprise a sensor component with a destabilized GFP under the control of a minimal thymidin kinase (TK) promoter and UAS element.

pM/hVDR and Gal4VP16 comprise hVDR or VP16, respectively, fused to the UAS binding region of Gal 4. VP16 is constitutively active, whereas hVDR requires ligand binding to function as transcriptions activator.

Sequences

Gal4 DBD:
SEQ ID NO: 1
Atgaagctactgtcttctatcgaacaagcatgcgatatttgccgactt
aaaaagctcaagtgctccaaagaaaaaccgaagtgcgccaagtgtctg
aagaacaactgggagtgtcgctactctcccaaaaccaaaaggtctccg
ctgactagggcacatctgacagaagtggaatcaaggctagaaagactg
gaacagctatttctactgatttttcctcgagaagaccttgacatgatt
ttgaaaatggattctttacaggatataaaagcattgttaacaggatta
tttgtacaagataatgtgaataaagatgccgtcacagatagattggct
tcagtggagactgatatgcctctaacattgagacagcatagaataagt
gcgacatcatcatcggaagagagtagtaacaaaggtcaaagacagttg
actgtatcg
hVDR LBD:
SEQ ID NO: 2
aagcggaaggaggaggaggccttgaaggacagtctgcggcccaagctg
tctgaggagcagcagcgcatcattgccatactgctggacgcccaccat
aagacctacgaccccacctactccgacttctgccagttccggcctcca
gttcgtgtgaatgatggtggagggagccatccttccaggcccaactcc
agacacactcccagcttctctggggactcctcctcctcctgctcagat
cactgtatcacctcttcagacatgatggactcgtccagcttctccaat
ctggatctgagtgaagaagattcagatgacccttctgtgaccctagag
ctgtcccagctctccatgctgccccacctggctgacctggtcagttac
agcatccaaaaggtcattggctttgctaagatgataccaggattcaga
gacctcacctctgaggaccagatcgtactgctgaagtcaagtgccatt
gaggtcatcatgttgcgctccaatgagtccttcaccatggacgacatg
tcctggacctgtggcaaccaagactacaagtaccgcgtcagtgacgtg
accaaagccggacacagcctggagctgattgagcccctcatcaagttc
caggtgggactgaagaagctgaacttgcatgaggaggagcatgtcctg
ctcatggccatctgcatcgtctccccagatcgtcctggggtgcaggac
gccgcgctgattgaggccatccaggaccgcctgtccaacacactgcag
acgtacatccgctgccgccacccgcccccgggcagccacctgctctat
gccaagatgatccagaagctagccgacctgcgcagcctcaatgaggag
cactccaagcagtaccgctgcctctccttccagcctgagtgcagcatg
aagctaacgccccttgtgctcgaagtgtttggcaatgagtctcctga
4 × UAS
SEQ ID NO: 3
Caaggcggagtactgtcctccgggctggcggagtactgtcctccggca
aggtcggagtactgtcctccgacactagaggtcggagtactgtcctcc
gacg
Minimal TK promoter
SEQ ID NO: 4
Gtggccgccccgactgcatctgcgtgttcaaattcgccaatgacaaga
cgctgggcggggtttgtgtcatcatagaactaaagacatgcaaatata
tttcttccggggacaccgccagcaaacgcgagcaacgggccacgggga
tgaagcag
d2eGFP
SEQ ID NO: 5
atggtgagcaagggcgaggagctgttcaccggggtggtgcccatcctg
gtcgagctggacggcgacgtaaacggccacaagttcagcgtgtccggc
gagggcgagggcgatgccacctacggcaagctgaccctgaagttcatc
tgcaccaccggcaagctgcccgtgccctggcccaccctcgtgaccacc
ctgacctggggcgtgcagtgcttcagccgctaccccgaccacatgaag
cagcacgacttcttcaagtccgccatgcccgaaggctacgtccaggag
cgcaccatcttcttcaaggacgacggcaactacaagacccgcgccgag
gtgaagttcgagggcgacaccctggtgaaccgcatcgagctgaagggc
atcgacttcaaggaggacggcaacatcctggggcacaagctggagtac
aactacatcagccacaacgtctatatcaccgccgacaagcagaagaac
ggcatcaaggccaacttcaagatccgccacaacatcgaggacggcagc
gtgcagctcgccgaccactaccagcagaacacccccatcggcgacggc
cccgtgctgctgcccgacaaccactacctgagcacccagtccgccctg
agcaaagaccccaacgagaagcgcgatcacatggtcctgctggagttc
gtgaccgccgccgggatcactctcggcatggacgagctgtacaagaag
RIR (T2-2. generation)
SEQ ID NO: 6
agtgtatgtaaacttctgacccactgggaatgtgatgaaagaaataaa
agctgaaatgaatcattctctctactattattctgatatttcacattc
ttaaaataaagtggtgatcctaactgacctaagacagggaatttttac
taggattaaatgtcaggaattgtgaaaaagtgagtttaaatgtatttg
gctaaggtgtatgtaaacttccgacttcaactg
LIR (T2-2. generation)
SEQ ID NO: 7
cagttgaagtcggaagtttacatacacttaagttggagtcattaaaac
tcgtttttcaactactccacaaatttcttgttaacaaacaatagtttt
ggcaagtcagttaggacatctactttgtgcatgacacaagtcattttt
ccaacaattgtttacagacagattatttcacttataattcactgtatc
acaattccagtgggtcagaagtttacatacact

Items

The following items define preferred embodiments of the present invention.

Item 1. A method for evaluating the effect of an agent in a tissue of an animal comprising

a. providing a transgenic animal, comprising at least one nucleic acid sequence, wherein

i. said at least one nucleic acid sequence encodes a reporter polypeptide or part thereof, and/or

ii. an additional nucleic acid sequence encodes a fusion polypeptide, comprising a nuclear receptor or part thereof coupled to a DNA binding domain, or the transcriptional or translational products of said additional nucleic acid sequence,

b. administering said agent to said animal, and

c. evaluating the transcriptional and/or translational expression product of the nucleic acid sequence encoding the reporter polypeptide,

wherein an alteration of said expression product prior to and after step (b) is indicative of an effect on said tissue.

Item 2. The method according to Item 1, wherein said agent is any physical or chemical agent.

Item 3. The method according to Item 1, wherein said agent is a pharmaceutical composition, cosmetic, drug, xenobiotic compound, food composition, sugar, lipid, protein, dietary supplement, radiation, electrical stimuli.

Item 4. The method according to Item 3, wherein said agent is in the form of solutions, crèmes, lotions, gels, microparticles, nanoparticles.

Item 5. The method according to any of the preceding, wherein said tissue is selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and tumours.

Item 6. The method according to any of the preceding, wherein said tissue is skin

Item 7. The method according to any of the preceding, wherein said tissue is epidermis.

Item 8. The method according to any of the preceding, wherein said tissue is dermis.

Item 9. The method according to any of the preceding, wherein said animal is selected from the group consisting of human, non-human primates, pig, minipig, micropig, mouse, rat and rodent.

Item 10. The method according to any of the preceding, wherein said animal is a human.

Item 11. The method according to any of the preceding, wherein said transgenic animal is a pig.

Item 12. The method according to any of the preceding, wherein said transgenic animal is a mouse.

Item 13. The method according to any of the preceding, wherein said reporter polypeptide or fragment thereof comprises a detectable product.

Item 14. The method according to Item 13, wherein said reporter polypeptide or fragment thereof comprises a visually, optically or autoradiographically detectable product.

Item 15. The method according to any of the preceding, wherein said reporter polypeptide is selected from the group consisting of β-galactosidase, HcRed, DsRed, DsRed monomer, ZsGreen, AmCyan, ZsYellow, fire fly luciferase, lac Z, renilla luciferase, SEAP, enhanced green fluorescent protein (eGFP), d2EGFP, enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, human alpha-1-antitrypsin (hAAT) and/or fragments, modifications or functional variants thereof.

Item 16. The method according to any of the preceding, wherein said reporter polypeptide is β-galactosidase.

Item 17. The method according to any of the preceding, wherein said evaluation comprises detection by any technique selected from the group consisting of enzymatic and spectroscopic assays, confocal and multiphoton fluorescent microscopy, western blotting, imunostaining, Enzyme-linked immunosorbent assay (ELISA) as well as nucleic acid detection techniques such as northern blotting, southern blotting, polymerase chain reaction, primer extension and DNA array technologies.

Item 18. The method according to any of the preceding, wherein said nucleic acid sequence encoding a reporter polypeptide is preceded by a promoter.

Item 19. The method according to Item 18, wherein said promoter is a heterologous promoter.

Item 20. The method according to Item 18, wherein said promoter is an inducible promoter.

Item 21. The method according to Item 18, wherein said promoter is thymidin kinase promoter.

Item 22. The method according to any of the preceding, wherein said promoter further comprises an enhancer element.

Item 23. The method according to Item 22, wherein said enhancer element is selected from the group consisting of the yeast UASgaI enhancer and the bacterial LexA binding site.

Item 24. The method according to Item 23, wherein said enhancer element is yeast UASgaI enhancer.

Item 25. The method according to any of the preceding, wherein said fusion polypeptide comprises a nuclear receptor or part thereof inserted within, and/or at the N-terminus and/or C-terminus of a DNA binding domain or part thereof.

Item 26. The method according to any of the preceding, wherein said fusion polypeptide comprises a nuclear receptor or part thereof inserted at the C-terminus of a DNA binding domain or part thereof.

Item 27. The method according to any of the preceding, wherein expression of said fusion polypeptide promotes expression of said reporter polypeptide.

Item 28. The method according to any of the preceding, wherein said additional nucleic acid sequence is preceded by a promoter.

Item 29. The method according to Item 28, wherein said promoter is an inducible promoter.

Item 30. The method according to Item 28, wherein said additional nucleic acid sequence encoding a nuclear receptor coupled to a DNA binding domain is expressed from a tissue-specific promoter.

Item 31. The method according to Item 30, wherein said tissue-specific promoter is specific for a tissue selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and/or tumours.

Item 32. The method according to Item 30, wherein said promoter is a skin-specific promoter.

Item 33. The method according to Item 28, wherein said promoter is keratin 14 enhancer/promoter.

Item 34. The method according to Item 28, wherein said promoter comprises enhancer elements.

Item 35. The method according to Item 28, wherein said promoter comprises a light-inducible sequence.

Item 36. The method according to Item 28, wherein said promoter comprises a chemically inducible sequence.

Item 37. The method according to any of the preceding, wherein said nuclear receptor or part thereof comprise at least one fragment of a ligand binding domain of a nuclear receptor.

Item 38. The method according to any of the preceding, wherein said nuclear receptor is Thyroid hormone receptor-α (TRα; NR1A1, THRA), Thyroid hormone receptor-β (TRβ; NR1A2, THRB), Retinoic acid receptor-α (RARα; NR1B1, RARA), Retinoic acid receptor-β (RARβ; NR1B2, RARB), Retinoic acid receptor-γ (RARγ; NR1B3, RARG), Peroxisome proliferator-activated receptor-α (PPARα; NR1C1, PPARA), Peroxisome proliferator-activated receptor-β/δ (PPARβ/δ; NR1C2, PPARD), Peroxisome proliferator-activated receptor-γ (PPARγ; NR1C3, PPARG), Rev-ErbAα (Rev-ErbAα; NR1D1), Rev-ErbAβ (Rev-ErbAβ; NR1D2), RAR-related orphan receptor-α (RORα; NR1F1, RORA), RAR-related orphan receptor-β (RORβ; NR1F2, RORB), Liver X receptor-α (LXRα; NR1H3), Liver X receptor-β (LXRβ; NR1 H2), Farnesoid X receptor (FXR; NR1H4), Vitamin D receptor (VDR; NR1I1, VDR) (vitamin D), Pregnane X receptor (PXR; NR1I2), Constitutive androstane receptor (CAR; NR1I3), Hepatocyte nuclear factor-4-α (HNF4α; NR2A1, HNF4A), Hepatocyte nuclear factor-4-γ (HNF4γ; NR2A2, HNF4G), Retinoid X receptor-α (RXRα; NR2B1, RXRA), Retinoid X receptor-β (RXRβ; NR2B2, RXRB), Retinoid X receptor-γ (RXRγ; NR2B3, RXRG), Testicular receptor 2 (TR2; NR2C1), Testicular receptor 4 (TR4; NR2C2), Human homologue of the Drosophila tailless gene (TLX; NR2E1), Photoreceptor cell-specific nuclear receptor (PNR; NR2E3), Chicken ovalbumin upstream promoter-transcription factor I (COUP-TFI; NR2F1), Chicken ovalbumin upstream promoter-transcription factor II (COUP-TFII; NR2F2), 6: V-erbA-related (EAR-2; NR2F6), Estrogen receptor-α (ERα; NR3A1, ESR1), Estrogen receptor-β (ERβ; NR3A2, ESR2), Estrogen related receptor-α (ERRα; NR3B1, ESRRA), Estrogen related receptor-β (ERRβ; NR3B2, ESRRB), Estrogen related receptor-γ (ERRγ; NR3B3, ESRRG), Glucocorticoid receptor (GR; NR3C1) (Cortisol), Mineralocorticoid receptor (MR; NR3C2) (Aldosterone), Progesterone receptor (PR; NR3C3, PGR) (Sex hormones: Progesterone), Androgen receptor (AR; NR3C4, AR) (Sex hormones: Testosterone), Nerve Growth factor IB (NGFIB; NR4A1), Nuclear receptor related 1 (NURR1; NR4A2), Neuron-derived orphan receptor 1 (NOR1; NR4A3), Steroidogenic factor 1 (SF1; NR5A1), Liver receptor homolog-1 (LRH-1; NR5A2), Germ cell nuclear factor (GCNF; NR6A1), DAX1 (Dosage-sensitive sex reversal, adrenal hypoplasia critical region, on chromosome X, gene 1 (NR0B1)), Small heterodimer partner (SHP; NR0B2) or Nuclear receptors with two DNA binding domains (2DBD-NR).

Item 39. The method according to any of the preceding, wherein said nuclear receptor is selected from the group consisting of vitamin D receptor, Liver X receptors, Retinoic Acid receptor, Retinoid X receptor, promiscuous pregnane X receptor and peroxisome proliferation activation receptors (PPARs), including PPARα, PPARβ/δ, PPARγ.

Item 40. The method according to any of the preceding, wherein said nuclear receptor is selected from the group consisting of PPARs.

Item 41. The method according to any of the preceding, wherein said nuclear receptor is PPARδ.

Item 42. The method according to any of the preceding, wherein said nuclear receptor is promiscuous pregnane X receptor.

Item 43. The method according to any of the preceding, wherein said DNA binding domain is selected from the group consisting of GAL4 DNA binding domain and LexA DNA binding domain.

Item 44. The method according to any of the preceding, wherein said administration comprises oral, including buccal and sublingual, rectal, nasal, topical, pulmonary, vaginal, or parenteral, including intramuscular, intraarterial, intrathecal, subcutaneous and intravenous administration or administration by inhalation or insufflation.

Item 45. The method according to any of the preceding, wherein said administration topical administration.

Item 46. The method according to any of the preceding, wherein said administration pulmonary administration.

Item 47. The method according to any of the preceding, wherein said expression product comprise RNA and/or polypeptide.

Item 48. The method according to any of the preceding, wherein said evaluation of the transcriptional and/or translational products is performed in the live animal.

Item 49. The method according to any of the preceding, wherein said evaluation of the transcriptional and/or translational products is performed without removing the tissue from the live animal.

Item 50. The method according to any of the preceding, wherein said evaluation of the transcriptional and/or translational products is performed on a sample removed from the animal.

Item 51. The method according to Item 50, wherein said sample is selected from the group consisting of skin tissue, including epidermal and dermal tissue, breast tissue, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and/or sweat.

Item 52. The method according to any of the preceding, further comprising a repeating of administering the agent to the tissue.

Item 53. The method according to any of the preceding, further comprising at least one additional evaluation step.

Item 54. The method according to Item 53, wherein the evaluation steps are separated by at least 1, 2, 3, 4, 5, 10, 20, 30, 60, 180, 365, or 700 days.

Item 55. A method for testing a compound for the ability to alter the effects of an agent in a tissue of an animal comprising

a. administering said compound to a transgenic animal comprising at least one nucleic acid sequence, wherein

i. said at least one nucleic acid sequence encodes a reporter polypeptide or part thereof, and/or

ii. an additional nucleic acid sequence encodes a fusion polypeptide, comprising a nuclear receptor or part thereof coupled to a DNA binding domain, or the transcriptional or translational products of said additional nucleic acid sequence,

b. administering said agent to said transgenic animal, and

c. evaluating the transcriptional and/or translational expression product of the nucleic acid sequence encoding the reporter polypeptide,

wherein a difference in the amount of said expression product in the presence and absence of said compound is indicative of said compound being able to alter the effect of said agent in said tissue.

Item 56. The method according to Item 55, as defined in any of Item 2 to Item 54.

Item 57. The method according to any of Item 55 and Item 56, wherein said compound is any physical or chemical agent.

Item 58. The method according to Item 57, wherein said compound is a pharmaceutical composition, cosmetic, drug, xenobiotic compound, food composition, sugar, lipid, dietary supplement, radiation or electrical stimuli.

Item 59. The method according to any of Item 57 and Item 58, wherein said compound is in the form of solutions, crèmes, lotions, gels, microparticles, nanoparticles.

Item 60. The method according to any of Item 55 and Item 59, wherein said compound is a sunlotion

Item 61. The method according to any of Item 55 to Item 60, wherein said agent is radiation

Item 62. The method according to Item 61, wherein said agent is UV-radiation.

Item 63. A transgenic animal comprising at least one nucleic acid sequence, wherein

i. said at least one nucleic acid sequence encodes a reporter polypeptide or part thereof, and/or

ii. an additional nucleic acid sequence encodes a fusion polypeptide, comprising a nuclear receptor or part thereof coupled to a DNA binding domain, or the transcriptional or translational products of said additional nucleic acid sequence

Item 64. The transgenic animal according to Item 63 for evaluating an agent for its effect on a tissue.

Item 65. The transgenic animal according to Item 63, wherein said animal is selected from the group consisting of pig, mouse, rat, rodent, dog, monkey, guinea pig, minipig and micropig.

Item 66. The transgenic animal according to Item 63, wherein said animal is pig.

Item 67. The transgenic animal according to Item 63, wherein said animal is mouse.

Item 68. The transgenic animal according to any of Item 63 to Item 67, wherein said reporter polypeptide is selected from the group consisting of β-galactosidase, HcRed, DsRed, DsRed monomer, ZsGreen, AmCyan, ZsYellow, fire fly luciferase, renilla luciferase, SEAP, EGFP, EBFP, EYFP, d2EGFP and GFPuv, cyan, green yellow, red, and far red Reef Coral Fluorescent Protein and/or fragments, modifications or functional variants thereof.

Item 69. The transgenic animal according to any of Item 63 to Item 68, wherein said reporter polypeptide is β-galactosidase or a fragment or functional variant thereof.

Item 70. The transgenic animal according to any of Item 63 to Item 69, wherein said nuclear receptor is selected from the group consisting of vitamin D receptor, Liver X receptors, promiscuous pregnane X receptor and PPARs, or a fragment thereof.

Item 71. The transgenic animal according to any of Item 63 to Item 70, wherein said DNA binding domain is selected from the group consisting of GAL4 DNA binding domain and LexA DNA binding domain.

Item 72. The transgenic pig according to Item 66, comprising at least one nucleic acid sequence, wherein

a. said at least one nucleic acid sequence encodes β-galactosidase or part thereof, and/or

b. an additional nucleic acid sequence encodes a fusion polypeptide, comprising PPARδ or part thereof coupled to yeast GAL4 DNA binding domain, or the transcriptional or translational products of said additional nucleic acid sequence.

Item 73. The transgenic animal according to any of Item 63 to Item 72, for determining in vivo the activation of nuclear receptors due to the production of endogenous agonists.

Item 74. The transgenic animal according to Item 73, wherein said agonists are generated during normal development of the skin

Item 75. The transgenic animal according to Item 73, wherein said endogenous agonists are generated during the development of a disease, such as psoriasis, different cancer types and/or other hyperproliferative diseases.

Item 76. The transgenic animal according to Item 75, wherein said disease is psoriasis.

Item 77. The transgenic animal according to any of Item 63 to Item 76, for determining penetration of an agent in situ in a tissue and/or the activation of nuclear receptors by an agent as defined in any of Item 2 to Item 4.

Item 78. A cell line derived from the transgenic animal according to any of Item 63 to Item 77.

1. A non-human transgenic animal comprising
i. at least one nucleic acid sequence encoding a nuclear receptor or part thereof and a DNA binding domain or part thereof, and
ii. at least one nucleic acid sequence encoding a detectable reporter nucleic acid transcript and/or reporter polypeptide or part thereof, wherein said nucleic acid further comprises at least one binding site for a polypeptide comprising a DNA binding domain and/or
iii. the transcriptional or translational products of any of said nucleic acid sequences.

2. The transgenic animal according to claim 1, wherein said transgenic animal is a pig.

3. The transgenic animal according to claim 1, wherein said non-human transgenic animal is selected from the group consisting of pig, minipig, micropig, mouse, rat, non-human primate and rodent.

4. The transgenic animal according to claim 1, comprising
a. at least one nucleic acid sequence encoding a fusion polypeptide, comprising PPARδ or part thereof coupled to yeast GAL4 DNA binding domain and/or
b. at least one nucleic acid sequence encoding β-galactosidase or part thereof.

5. The transgenic animal according to claim 1, wherein said nuclear receptor or part thereof comprise a ligand binding domain of a nuclear receptor or a fragment thereof.

6. The transgenic animal according to claim 1, wherein said nuclear receptor is Thyroid hormone receptor-α (TRα; NR1A1, THRA), Thyroid hormone receptor-β (TRβ; NR1A2, THRB), Retinoic acid receptor-α (RARα; NR1B1, RARA), Retinoic acid receptor-β (RARβ; NR1B2, RARB), Retinoic acid receptor-γ (RARγ; NR1B3, RARG), Peroxisome proliferator-activated receptor-α (PPARα; NR1C1, PPARA), Peroxisome proliferator-activated receptor-β/δ (PPARβ/δ; NR1C2, PPARD), Peroxisome proliferator-activated receptor-γ (PPARγ; NR1C3, PPARG), Rev-ErbAα (Rev-ErbAα; NR1D1), Rev-ErbAβ (Rev-ErbAβ; NR1D2), RAR-related orphan receptor-α (RORα; NR1F1, RORA), RAR-related orphan receptor-β (RORβ; NR1F2, RORB), Liver X receptor-α (LXRα; NR1H3), Liver X receptor-β (LXRβ; NR1H2), Farnesoid X receptor (FXR; NR1H4), Vitamin D receptor (VDR; NR1I1, VDR) (vitamin D), Pregnane X receptor (PXR; NR1I2), Constitutive androstane receptor (CAR; NR1I3), Hepatocyte nuclear factor-4-α (HNF4α; NR2A1, HNF4A), Hepatocyte nuclear factor-4-γ (HNF4γ;
NR2A2, HNF4G), Retinoid X receptor-α (RXRα; NR2B1, RXRA), Retinoid X receptor-β (RXRβ; NR2B2, RXRB), Retinoid X receptor-γ (RXRγ; NR2B3, RXRG), Testicular receptor 2 (TR2; NR2C1), Testicular receptor 4 (TR4; NR2C2), Human homologue of the Drosophila tailless gene (TLX; NR2E1), Photoreceptor cell-specific nuclear receptor (PNR; NR2E3), Chicken ovalbumin upstream promoter-transcription factor I (COUP-TFI; NR2F1), Chicken ovalbumin upstream promoter-transcription factor II (COUP-TFII; NR2F2), 6: V-erbA-related (EAR-2; NR2F6), Estrogen receptor-α (ERα; NR3A1, ESR1), Estrogen receptor-β (ERβ; NR3A2, ESR2), Estrogen related receptor-α (ERRα; NR3B1, ESRRA), Estrogen related receptor-β (ERRβ; NR3B2, ESRRB), Estrogen related receptor-γ (ERRγ; NR3B3, ESRRG), Glucocorticoid receptor (GR; NR3C1) (Cortisol), Mineralocorticoid receptor (MR; NR3C2) (Aldosterone), Progesterone receptor (PR; NR3C3, PGR) (Sex hormones: Progesterone), Androgen receptor (AR; NR3C4, AR) (Sex hormones: Testosterone), Nerve Growth factor IB (NGFIB; NR4A1), Nuclear receptor related 1 (NURR1; NR4A2), Neuron-derived orphan receptor 1 (NOR1; NR4A3), Steroidogenic factor 1 (SF1; NR5A1), Liver receptor homolog-1 (LRH-1; NR5A2), Germ cell nuclear factor (GCNF; NR6A1), DAX1 (Dosage-sensitive sex reversal, adrenal hypoplasia critical region, on chromosome X, gene 1 (NR0B1)), Small heterodimer partner (SHP; NR0B2) or Nuclear receptors with two DNA binding domains (2DBD-NR).

7. The transgenic animal according to claim 1, wherein said nuclear receptor is selected from the group consisting of vitamin D receptor, Liver X receptors, Retinoic Acid receptor, Retinoid X receptor, promiscuous pregnane X receptor and peroxisome proliferation activation receptors (PPARs), including PPARα, PPARβ/δ, PPARγ.

8. The transgenic animal according to claim 1, wherein said DNA binding domain is GAL4 DNA-binding domain, LexA DNA-binding domain, and/or a part thereof.

9. The transgenic animal according to claim 1, wherein said nuclear receptor or part thereof and a DNA binding domain or part thereof are expressed from an inducible and/or a tissue-specific promoter.

10. The transgenic animal according to claim 0, wherein said tissue-specific promoter is specific for a tissue selected from the group consisting of skin, epidermis, dermis, hypodermis, fat, thymus, gut, small intestine, large intestine, stomach, muscle, pancreas, heart muscle, skeletal muscle, smooth muscle, liver, lung, brain, cornea and/or tumours.

11. The transgenic animal according to claim 0, wherein said promoter is keratin 14 enhancer/promoter.

12. The transgenic animal according to claim 1, wherein said nuclear receptor or part thereof and a DNA binding domain or part thereof are physically or chemically coupled.

13. The transgenic animal according to claim 1, wherein said nucleic acid sequence encoding a detectable reporter nucleic acid transcript and/or reporter polypeptide or part thereof further comprises at least one yeast Gal4 upstream activation sequence (UAS_{gal}), bacterial LexA binding site and/or a part thereof.

14. The transgenic animal according to claim 1, wherein said nucleic acid sequence encoding a detectable reporter transcript or polypeptide is expressed from a heterologous and/or inducible promoter.

15. The transgenic animal according to claim 1, wherein expression of said nuclear receptor or part thereof and a DNA binding domain or part thereof promotes expression of said reporter polypeptide in the presence of a ligand specific for said nuclear receptor.

16. The transgenic animal according to claim 15, wherein said reporter transcript, polypeptide or fragment thereof comprises a visually, optically or autoradiographically detectable product.

17. The transgenic animal according to claim 16, wherein said reporter polypeptide is selected from the group consisting of β-galactosidase, HcRed, DsRed, DsRed monomer, ZsGreen, AmCyan, ZsYellow, fire fly luciferase, lac Z, renilla luciferase, SEAP, enhanced green fluorescent protein (eGFP), d2EGFP, enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, human alpha-1-antitrypsin (hAAT) and/or fragments, modifications or functional variants thereof.

18. The transgenic animal according to claim 16, wherein said reporter polypeptide is β-galactosidase.

19. The transgenic animal according to claim 15, wherein said expression of said reporter transcript or polypeptide is detectable by any technique selected from enzymatic or spectroscopic assays, confocal or multiphoton fluorescent microscopy, western blotting, imunostaining, Enzyme-linked immunosorbent assay (ELISA) as well as nucleic acid detection techniques such as northern blotting, southern blotting, polymerase chain reaction, primer extension and DNA array technologies.

20. A method for evaluating the effect of an agent on the activity of a nuclear receptor in a tissue of a non-human animal, said method comprising
c. providing a non-human transgenic animal as defined in claim 1,
d. administering an agent to said transgenic animal, and
e. detecting the expression of said nucleic acid sequence encoding a reporter nucleic acid transcript and/or reporter polypeptide or part thereof in said animal,
wherein the expression upon administration of said agent is indicative of the effect of said agent on the activity of a nuclear receptor in said tissue.

21. A method for testing a compound for the ability to alter an effect of an agent on the activity of a nuclear receptor in a tissue of a non-human animal comprising
f. providing a non-human transgenic animal as defined in claim 1,
g. administering said compound to said transgenic animal,
h. administering said agent to said transgenic animal, and
i. detecting the expression of said nucleic acid sequence encoding a reporter nucleic acid transcript and/or reporter polypeptide or part thereof in said animal,
wherein the expression upon administration of said agent is indicative of the effect of said agent on the activity of a nuclear receptor in said tissue.

22. (canceled)

23. The method according to claim 20, wherein in the presence of said agent compared with the absence of said agent
a. an increase in the expression of said reporter transcript or polypeptide is indicative of a stimulatory effect of said agent on the activity of said nuclear receptor,
b. a decrease in the expression of said reporter transcript or polypeptide is indicative of an inhibitory effect of said agent on the activity of said nuclear receptor, and
c. an unchanged expression of said reporter transcript or polypeptide is indicative of said agent having no or little effect on the activity of said nuclear receptor.

24. The method according to claim 21, wherein in the presence of said compound compared with the absence of said compound
a. an increase in the effect of said agent on the expression of said reporter transcript or polypeptide is indicative of a stimulatory effect of said compound on the effect of said agent on the activity of said nuclear receptor,
b. a decrease in the effect of said agent on the expression of said reporter transcript or polypeptide is indicative of an inhibitory effect of said compound on the effect of said agent on the activity of said nuclear receptor, and
c. a little or unchanged effect of said agent on the expression of said reporter transcript or polypeptide is indicative of said compound having no or little effect on the effect of said agent on the activity of said nuclear receptor.

25-30. (canceled)

31. The method according to claim 21, wherein said agent or compound is in the form of solutions, crèmes, lotions, gels, microparticles, and/or nanoparticles.

32-33. (canceled)

34. The method according to claim 20, wherein said detection of the transcriptional and/or translational products is performed in the live animal.

35. (canceled)

36. The method according to claim 20, wherein said detection of a transcriptional and/or translational reporter product is performed on a tissue sample removed from the animal.

37. The method according to claim 20, wherein said tissue is selected from the group consisting of skin, epidermis, dermis, hypodermis, breast, fat, thymus, gut, small intestine, large intestine, stomach, muscle, pancreas, heart muscle, skeletal muscle, smooth muscle, liver, lung, brain, cornea and tumours, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cell, and body fluids, such as sputum, urine, blood and/or sweat.

38. (canceled)

39. A cell line derived from the transgenic animal according to claim 1.

40. A transgenic non-human oocyte, sperm cell, blastocyst, embryo, fetus, donor cell, or cell nucleus derived from the transgenic non-human animal as defined in claim 1, and/or
a transgenic non-human oocyte, sperm cell, blastocyst, embryo, fetus, donor cell, or cell nucleus, wherein the transgenic genome comprises
i. at least one nucleic acid sequence encoding a nuclear receptor or part thereof and a DNA binding domain or part thereof, and
ii. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or
iii. the transcriptional or translational products of any of said nucleic acid sequences.

41. A method of producing a transgenic non-human animal according to claim 1, a cell line, an oocyte, sperm cell, blastocyst, embryo, fetus, donor cell, or cell nucleus derived from the transgenic animal according to claim 1 comprising the steps of
i. providing a donor cell,
ii. genetically modifying the donor cell of i) by inserting
a. at least one at least one nucleic acid sequence encoding a nuclear receptor or part thereof and a DNA binding domain or part thereof, and
b. at least one nucleic acid sequence encoding a detectable reporter nucleic acid transcript and/or reporter polypeptide or part thereof, wherein said nucleic acid further comprise at least one binding site for a polypeptide comprising a DNA binding domain and/or
c. the transcriptional or translational products of any of said nucleic acid sequences,
iii. transferring the modified genome of the donor cell obtained in ii) into a host cell,
iv. obtaining a reconstructed embryo forming an embryo
v. culturing said embryo; and
vi. transferring said cultured embryo to a host mammal such that the embryo develops into a genetically modified fetus,
wherein said genetically modified embryo is produced by nuclear transfer comprises steps i) to v),
wherein said genetically modified blastocyst is produced by nuclear transfer comprises steps i) to vi),
wherein said genetically modified fetus is produced by nuclear transfer comprises steps i) to vi).

42. Use of a transgenic animal as defined in claim 1, cell line derived from the transgenic animal according to claim 1, and/or an oocyte, sperm cell, blastocyst, embryo, fetus, donor cell, or cell nucleus derived from the non-human transgenic animal as defined in claim 1 for evaluating the activity of a nuclear receptor.

43-47. (canceled)
